(19) United States
(12) Patent Application Publication
(10) Pub. No.: US 2016/0230143 A1
(43) Pub. Date: Aug. 11, 2016

(54) CHEMICALLY DEFINED CULTURE MEDIUM FOR STEM CELL MAINTENANCE AND DIFFERENTIATION

(71) Applicant: THE U.S.A., AS REPRESENTED BY THE SECRETARY, DEPARTMENT OF HEALTH & HUMAN SERVICES, Rockville, MD (US)

(72) Inventors: Guokai CHEN, Rockville, MD (US); Yongshun LIN, Gaithersburg, MD (US)

(21) Appl. No.: 15/023,221

(22) PCT Filed: Sep. 19, 2014
(86) PCT No.: PCT/US2014/056485

§ 371 Date: Mar. 18, 2016

Related U.S. Application Data
(60) Provisional application No. 61/879,840, filed on Sep. 19, 2013.

(51) Int. Cl. C12N 5/077; A61K 35/34

(52) U.S. Cl. CPC C12N 5/0657; A61K 35/34; C12N 2501/33; C12N 2501/115; C12N 2500/25; C12N 2501/15; C12N 2500/32; C12N 2500/38; C12N 2500/05; C12N 2501/91; C12N 2506/45

(57) ABSTRACT

The present invention is directed to a low protein medium for the culture of pluripotent stem cells comprising one or more polysaccharides. The medium of the present invention can support cell culture objectives, for example cell survival, maintenance, passaging, proliferation, pluripotency, cloning, differentiation and induced pluripotent stem (iPS) cell derivation.

### CROSS-REFERENCE TO RELATED APPLICATIONS

The present application claims priority to, and the benefit under 35 U.S.C. §119(e) of, U.S. provisional patent application No. 61/879,840, filed Sep. 19, 2013, entitled “Chemically Defined Culture Medium for Stem Cell Maintenance and Differentiation”. The entire teachings of this application are incorporated herein by reference.

### GOVERNMENT FUNDING

Research supporting this application was carried out by the United States of America, as represented by the Secretary, Department of Health and Human Services.

### BACKGROUND OF THE INVENTION

Human pluripotent stem cells (hPSCs), including human embryonic stem cells (hESCs) and human induced pluripotent stem cells (iPSCs), can be propagated indefinitely while still retaining the capacity to differentiate into all somatic cell types, and are a potentially inexhaustible supply of human cells. E8 medium is a chemically defined cell culture medium developed in the laboratory of Dr. James Thomson (University of Wisconsin-Madison), and is one of the most widely published/used feeder-free cell culture medium for human ES cells and iPS cells. However, a problem remains that stem cells cannot survive at confluent or high density in low protein medium such as E8. High density culture is often used in large scale stem cell expansion and differentiation. The capacity to sustain survival at high density is critical for maintaining consistent stem cell cultures and avoiding the development of abnormal stem cells, and for proper stem cell differentiation.

Cardiovascular/heart disease is the number one cause of death. At the same time, heart is often the main target of toxicity of drugs. Many treatments require organ transplantation or cell based therapies. It is advantageous to transplant healthy cardiac cells derived from the patient iPSCs. High quality cardiac cells are essential for excluding high-risk drugs. Patient specific cardiac cells could also be used to test drug dose for personalized therapies. It is important to generate large quantities of patient specific cardiac cells that could be used in therapy, diagnosis and screening. Cardiomyocyte differentiation from ESC/iPSCs is usually conducted in high-density culture, and conventional procedures usually use albumin containing or other serum-product containing high protein culture conditions. Cardiomyocyte differentiation in chemically defined low-protein culture has not been realized until the current invention.

### SUMMARY OF THE INVENTION

The present invention is based, in part, on the surprising finding that addition of certain factors to low protein culture medium enhances pluripotent stem cell survival and differentiation efficiency. Some aspects of the present invention can be used to produce stem cells. Related aspects of the invention can be used to induce and/or otherwise improve differentiation of cardiomyocytes.

In certain aspects, the present invention provides a low protein culture medium with defined chemical components that allows pluripotent stem cell maintenance and differentiation. In related aspects, the present invention also provides for production of high quality cardiac cells from human embryonic and induced pluripotent stem cells in chemically defined conditions.

In one aspect, the present invention features a low protein medium that supports the proliferation and differentiation of stem cells comprising one or more of a volume expander, a lipid mix and a growth factor modulator.

In one embodiment, the volume expander comprises one or more polysaccharides. In a related embodiment, the one or more polysaccharides is selected from the group consisting of trehalose, CM-Dextran or glycogen, beta-cyclodextrin, beta-hydrodextrin, N-Acetyl-Glucosamine, Methyl alpha-D-Glycogyrate, Methyl beta-D-glycogyrate, Dextrin 100K, Methyl-1-beta-cyclodextrin. In another embodiment, the volume expander is taurine or albumin.

In another embodiment, the lipid mix comprises arachadonic acid, cholesterol, DL-alpha-tocopherol acetate, ethyl alcohol 100%, linoleic Acid, linolenic Acid, myristic acid, oleic acid, palmitic acid, palmitoleic acid, Pluronic F-68, stearic acid and tween-80.

In another further embodiment, the growth factor modulator is selected from the group consisting of: heparin and heparan sulfate.

In one embodiment, the medium is albumin-free.

In another embodiment, the medium comprises water, salts, amino acids, vitamins, a carbon source, insulin, an FGF, selenium, transferrin, and one of TGF-β and NODAL, each in an amount sufficient to support pluripotent stem cell proliferation.

In another embodiment, the stem cell is a totipotent, pluripotent, multipotent, oligopotent or unipotent stem cell. In another further embodiment, the stem cell is an embryonic stem cell (ESCs), an induced pluripotent stem cell (iPSCs), a fetal stem cell or an adult stem cell. In a further related embodiment, the stem cell is a mammalian stem cell. In other further embodiments, the mammalian stem cell is a human stem cell. In still other further embodiments, the stem cell is a human embryonic stem cell or a human induced pluripotent stem cell.

In another embodiment, the medium can support proliferation at high density. In a further embodiment, high density is confluency of 70% or more.

In one embodiment, the low protein medium of any of the aspects described herein comprises a volume expander.

In one embodiment, the low protein medium of any of the aspects described herein comprises a lipid mix and a growth factor modulator.

In another aspect, the present invention features a method for culturing stem cells, the method comprising the steps of placing pluripotent stem cells on a matrix or in suspension; and contacting the cells with the low protein medium described in the aspects herein.

In another aspect, the present invention features a method for deriving an induced pluripotent stem cell, the method comprising the steps of placing the pluripotent stem cells on a matrix or in suspension; and contacting the cells with the low protein medium described in the aspects herein.

In one embodiment, the stem cell is a totipotent, pluripotent, multipotent, oligopotent or unipotent stem cell. In another embodiment, the stem cell is an embryonic stem cell (ESCs), an induced pluripotent stem cell (iPSCs), a fetal stem cell or an adult stem cell. In another further embodiment, the stem cell is a mammalian stem cell. In still another further embodiment, the mammalian stem cell is a human stem cell. In a further related embodiment, the stem cell is a human embryonic stem cell or a human induced pluripotent stem cell.

In another aspect, the present invention features a method for deriving a cardiac cell under defined conditions, the method comprising the step of culturing a pluripotent stem cell in the low protein medium described in the aspects herein. In one aspect, the present invention discloses a method of producing high quality cardiac cells from embryonic stem cells or IPSCs and the method involves using a low protein medium with heparin or heparin sulfate. In another aspect of the invention, a method of producing high quality cardiac cells from embryonic stem cells or IPSCs and the method involves using a low protein medium with heparin or heparin sulfate along with WNT inhibitors.

Another aspect of the invention provides a method for inducing differentiation of a cardiac cell in vitro, involving: obtaining a population of cells including a stem cell; and contacting the population of cells with a low protein medium including a volume expander, a lipid mix and/or a growth factor modulator in an amount sufficient to induce differentiation of a cardiac cell from a stem cell of the population.

In one embodiment, the medium includes at least about 0.3 μg/ml heparin or heparan sulfate. In another embodiment, the medium includes at least about 1 μg/ml heparin or heparan sulfate. In an additional embodiment, the medium includes heparin or heparan sulfate at about 1 μg/ml.

In another embodiment, the medium includes insulin.

In one embodiment, the contacting occurs on days 1-7 of culture.

An additional aspect of the invention provides a cardiac cell prepared by a method of the invention.

A further aspect of the invention provides a method for performing a cardiac toxicity assay that involves contacting a cardiac cell produced by a method of the invention with a drug, and monitoring the function and/or survival of the cardiac cell.

An additional aspect of the invention provides a method for transplanting a cardiac cell into a subject that involves obtaining a cardiac cell prepared by a method of the invention, and administering the cardiac cell to the subject.

In one embodiment, the cardiac cell is administered to the heart of the subject, optionally by injection. In a related embodiment, the subject is human.

In certain embodiments, the subject has heart disease.

In related embodiments, the population of cells obtained to produce the cardiac cell is a population of cells of the subject.

In one embodiment, the low protein medium comprises a lipid mix and a growth factor modulator.

In one embodiment of any one of the above aspects, one or more promoters of WNT signaling are added. In another embodiment of any one of the above aspects, one or more inhibitors of WNT signaling are added.

In one embodiment of any of the above aspects, one or more growth factors are removed from the low protein medium. In a further embodiment, the growth factors are selected from the group consisting of: TGFβ, FGF and insulin.

In another aspect, the present invention features a kit comprising a low protein medium that supports the proliferation and differentiation of stem cells comprising one or more of a volume expander, a lipid mix and a growth factor modulator.

In one embodiment, the kit further comprises directions for the use of the medium for culturing stem cells or deriving an induced pluripotent stem cell.

In one aspect of the present invention, a method of producing cardiac cells from stem cells is disclosed and the method comprises the use of heparin and/or heparin sulfate along with chemically defined media during the differentiation protocol.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “albumin-free medium” means that a medium does not contain albumin or an albumin replacement, or that it contains essentially no albumin or albumin replacement. For example, an “albumin-free medium” can contain less than about 10%, 9%, 8%, 7%, 6%, 5%, 4%, 3%, 2% or 1% serum, wherein the culturing capacity of the medium is still observed.

As used herein, the term “cardiomyocyte” is meant to include cardiomyocyte progenitor cells having the ability to become functional cardiomyocytes in the future, as well as fetal and adult cardiomyocytes at all stages of differentiation. Cardiomyocytes can be identified by one or more than one marker or index.

As used herein, the term “cell culture objective” is meant to refer to any desired outcome of cell culture. Examples of culture objectives include, but are not limited to, cell survival, maintenance, passaging, proliferation, pluripotency, cloning, differentiation and induced pluripotent stem (iPS) cell derivation.

As used herein, the term “cloning” refers to initiating clonal colonies by growing human ES cell colonies from single individual ES cells. “cloning efficiency” refers to the number of individualized cells that form new cell colonies divided by the number of individualized cells plated in culture. Cloning efficiency varies considerably depending on culture conditions.

As used herein, the term “high density” is meant to refer to a high density of cells in culture. In certain embodiments, high density refers to growing stem cells at >70%, for example 70%, 75%, 80%, 85%, 90%, 95%. In certain embodiments, “high density” refers to >90% cell confluence for several days.

As used herein, “low protein medium” means that a medium contains a low percentage of protein. For example, a “low protein medium” can contain less than about 10%, 9%, 8%, 7%, 6%, 5%, 4%, 3%, 2% or 1% serum, wherein the culturing capacity of the medium is still observed. In certain embodiments, the low protein medium is an albumin free medium.

As used herein, the term “passaging” is meant to refer to the process of dividing cells that have been cultivated in a culture vessel up to a certain density into aggregates, which are then placed into new culture vessels. These aggregates can contain any number of cells, typically between 100 to 1,000 cells, which readily initiate growth in culture.

As used herein, the term “pluripotent cell” means a cell capable of differentiating into cells of all three germ layers. Examples of pluripotent cells include embryonic stem cells and induced pluripotent stem (iPS) cells. As used herein, “iPS cells” refer to cells that are substantially genetically identical to their respective differentiated somatic cell of origin and display characteristics similar to higher potency cells, such as embryonic stem (ES) cells, as described herein. The cells can be obtained by reprogramming non-pluripotent (e.g. multipotent or somatic) cells.

As used herein, the term “pluripotent” or “pluripotency” is meant to refer to a cell's ability to differentiate into cells of all three germ layers. Pluripotent stem cells are meant to refer to cells which are capable of indefinite or long-term cell proliferation while remaining in an undifferentiated state in an in vitro culture, which retain normal karyotypes, and which have the ability to differentiate into all of three germ layers (ectoderm, mesoderm and endoderm) under appropriate conditions.

As used herein, the term “somatic cell reprogramming” is meant to refer to a process whereby somatic cells are reprogrammed to induced pluripotent stem cells (iPSCs).

The term “stem cell” is meant to refer to cells found in all multicellular organisms, that can divide (through mitosis) and differentiate into diverse specialized cell types and can self-renew to produce more stem cells. In mammals, there are two broad types of stem cells: embryonic stem cells, which are isolated from the inner cell mass of blastocysts, and adult stem cells, which are found in various tissues.

As used herein, the term “matrix” is meant to refer to a substrate that cells can be grown and cultured on. An exemplary matrix is MATRIGEL.

As used herein, the term “volume expander” is meant to refer to a factor that is added to the culture medium that supports cell proliferation and differentiation. In certain embodiments, the volume expander is a polysaccharide.

As used herein the term “lipid mix” is meant to refer to a chemically defined mixture of lipids for use in cell culture. In certain embodiments, the lipid mix comprises arachadonic acid, cholesterol, DL-alpha-tocopherol acetate, ethyl alcohol 100%, linoleic Acid, linolenic Acid, myristic acid, oleic acid, palmitic acid, palmitoleic acid, Pluronic F-68, stearic acid, tween-80. In certain exemplary embodiments, the lipid mix comprises arachadonic acid (2 mg/ml), cholesterol (220 mg/ml), DL-alpha-tocopherol acetate (70 mg/ml), ethyl alcohol 100%, linoleic Acid (10 mg/ml), linolenic acid (10 mg/ml), myristic acid (10 mg/ml), oleic acid 10 mg/ml), palmitic acid (10 mg/ml), palmitoleic acid (10 mg/ml), Pluronic F-68 (90000 mg/ml), stearic acid (10 mg/ml), tween-80 (2200 mg/ml). In further exemplary embodiments, the lipid mix is commercially available as Chemically Defined Lipid Concentrate (Cat#11905031) from Life Technologies.

About: As used herein, the term “about” means+/−10% of the recited value. Use of “about” is contemplated in reference to all ranges and values recited herein.

As used herein, the term “growth factor modulator” is meant to refer to a factor that is added to the culture medium that supports cell proliferation and differentiation. In certain embodiments, the growth factor modulator is heparin, heparan sulfate or dextran sulfate.

### DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1 shows the results of screening assays with indicated additives (trehalose, CM-dextran, lipid mix, heparin, as indicated).

FIG. 2 shows the results of screening assays with different additives (trehalose, CM-dextran, glycogen, albumin, lipid mix, heparin).

FIG. 3 shows cell proliferation and differentiation under normal (“good incubator”) and suboptimal (“bad incubator”) conditions. Additives are indicated (glycogen, glycogen+lipid+heparin).

FIG. 4 shows effects of different doses of heparin on over-confluent hESCs.

FIG. 5 shows effects of different doses of additives (glycogen, DM-Dextran, and Trehalose) on over-confluent hESCs.

FIG. 6 (a and b) shows cell proliferation and spontaneous differentiation at high density with trehalose and CM-dextran. In (a), H1 hESCs were cultured in E8 media to >90% confluence, and then continually cultured for 5 days in E8 media with indicated additives. Phase contrast images show the survival rates of the cells. Note that most of cell in control condition died and few cells regrow, while Trehalose and CM-Dextran treated cells survived in high confluence but some of them started to differentiate. In (b), qPCR of pluripotent and differentiated genes expression is shown for the samples in a.

FIG. 7 shows a model of relationships between cell death, self-renewal, and differentiation, in low and high density culture. The top panels are shown at 5 days after confluence, at 10× magnification. The schematic in the bottom panel shows the relationship between cell death, self-renewal and differentiation at low density (left) and high density (right) culture.

FIG. 8 shows that the newly identified factors (indicated in the figure as CM-Dextran, glycogen, trehalose, heparin, or in some combinations) suppressed cell death during cardiomyocyte differentiation using the Test 1 protocol. Cell survival at day 3 is shown.

FIG. 9 shows that the newly identified factors (dextran, glycogen, trehalose, heparin, alone or in some combinations) suppressed cell death during cardiomyocyte differentiation at Test 3 condition. Cell survival at day 3 is shown.

FIG. 10 are graphs that show the new factors affected specific cardiac gene expression during differentiation. Expression of Nkx.2, TBX5 and cTnT is shown as determined by qPCR.

FIGS. 11 a and b shows (a) new protocol based on E8 conditions for cardiac production and (b) cardiac marker cTnT (green) staining and FACS assay showed the efficiency of cardiomyocyte differentiation.

FIG. 12 shows that the new factors improved cardiomyocyte differentiation at new cardiac differentiation condition 1. H1 hESCs were differentiated in a new condition 1 protocol, as shown, and treated with the indicated factors (glycogen, heparin, glycogen+heparin and control), and the images of cells at day 18 are shown.

FIG. 13 shows that the new factors improved cardiomyocyte differentiation at new cardiac differentiation condition 2. H1 hESCs were differentiated in a new condition 2 protocol, as shown, and treated with the indicated factors (glycogen, heparin, glycogen+heparin and control), and the images of cells at day 18 are shown.

FIG. 14 shows the results of experiments screening for factors promoting cardiac differentiation. The indicated growth factors or inhibitors as shown on the x-axis were added at day 0-1 during cardiomyocyte differentiation. qPCR of cTnT expression suggested that Heparin significantly enhance cardiac derivation.

FIG. 15 is a graph that shows heparin is beneficial for cardiac differentiation with different WNT modulators as indicated (IWP2, IWR1, XAV939, KY). 1 ug/ml Heparin was added into media during cardiomyocyte differentiation using different WNT inhibitors as indicated. FAC assay was assessed using cTnT antibody to show the positive rate of cardiomyocytes after 10 days differentiation. The results demonstrated that Heparin promoted cardiomyocyte production with all different WNT inhibitors.

FIG. 16 is a set of panels that show heparin promotes cardiomyocyte differentiation in the presence or absence of WNT inhibitors. Concentrations of WNT inhibitor (IWP2) and heparin are indicated. 1 ug/ml Heparin was added into media during cardiomyocyte differentiation with/without WNT inhibition Immunofluorescence staining of cTnT antibody (green) show the positive cardiomyocytes after 10 days differentiation. The results indicated that Heparin promoted cardiomyocyte production even without WNT inhibitors (nuclei were stained by DAPI, blue).

FIG. 17 shows the results of microarray analysis that show heparin induced cardiac differentiation at different time points. mRNA was collected at indicated periods and submitted for microarray. The relative expression of cTnT from array data is shown.

FIG. 18 shows cardiomyocyte production with 1 ug/ml heparin treatment at different time periods. Time period is indicated below each panel.

FIG. 19 shows the effects of different dosages of heparin (0.3-30 ug/ml) on cardiomyocyte differentiation.

FIG. 20 shows a doxycycline (doxo) killing curve where cardiomyocytes were treated with doxo at indicated concentrations for two days.

FIG. 21 shows results of transplantation experiments. Cardiomyocytes carrying GFP derived from ND2 hiPSCs were injected into mouse surgery heart, the GFP labeled cells can be survived and detected in vivo several after injection.

FIG. 22 shows results that demonstrated the general function of heparin on different human ESC/iPSC lines. Specifically, the effect of heparin on cardiac differentiation was tested on the multiple indicated human ESC and iPSC lines. The percentages of Troponin T (CTNT) positive cells at day 10 were detected by FACS. Ctrl: control; ip: treat IWP2 at day 2-5; h3: treat heparin (3 ug/ml) at day 1-7; ip,h3: treat IWP2 at day 2-5 and heparin (3 ug/ml) at day 1-7.

### DETAILED DESCRIPTION OF THE INVENTION

Chemically defined culture medium has long been an ideal platform for stem cell research and applications. However, cell death which occurs when cells reach high density affects the maintenance and differentiation efficiency of stem cells. The treatments developed in undefined high protein media usually have to be adjusted or re-invented in defined low-protein conditions such as E8. It would be ideal to bridge the gap between the cells cultured under undefined high protein conditions and defined culture medium conditions. The present invention is based, in part, on the surprising finding that addition of certain factors to low protein culture medium enhances pluripotent stem cell survival and differentiation efficiency when the cells reach high density or when the conditions for differentiation require high density. The present invention provides a low protein medium that supports maintenance, proliferation or differentiation of pluripotent stem cells and comprises one or more polysaccharides. The present invention further provides a method for cardiac differentiation from stem cells using heparin and heparan sulfate to supplement the low protein medium.

### Pluripotent Stem Cells

Pluripotent cells, such as embryonic stem (ES) cells and induced pluripotent stem (iPS) cells, have the potential to differentiate into cells of all three primary germ layers (Thomson, et al., Science 282, 1145-1147 (1998)). Pluripotent stem cells exist only transiently during embryogenesis, and the cells in dish are an artifact of cell culture conditions. The remarkable developmental potential of pluripotent cells has proven useful for basic research and clinical application. Many basic methods for human pluripotent cell culture, such as growth media, plate coating, and other conditions, have been developed and refined (Ludwig et al., Nat. Biotechnol 24, 185-187 (2006); Ludwig et al., Nat. Methods 3, 637-646 (2006)). For example, while human ES cells were initially cultured in fetal bovine serum (FBS)-containing media on murine embryonic fibroblast (MEF) feeder cells, fully defined media as well as defined protein matrices are now available (Ludwig et al, Nat. Biotechnol 24, 185-187 (2006)). Pluripotent cell culture methods have evolved considerably. Several growth media were developed that provide basic nutrients and growth factors for survival and expansion of pluripotent cells and directly determine how cells grow and differentiate. TeSR was one of the first defined media that supports pluripotent cell maintenance in an undifferentiated state in the absence of feeder cells or conditioned medium through multiple culture passages (Ludwig et al., Nat. Methods 3, 637-646 (2006); U.S. Pat. No. 7,449,334, each of which is incorporated herein by reference as if set forth in its entirety). TeSR contains 18 components in addition to the basal medium DMEM/F12 that itself has 52 components. A complete list of ingredients of DMEM/F12 is set forth in Table 1, below. Although TeSR could be used to derive human ESCs in the complete absence of animal proteins, the inclusion of human serum albumin and human-sourced matrix proteins makes those conditions prohibitively expensive, impractical for routine use, are inconsistent from batch to batch, and not truly completely defined.

TABLE 1

| Inorganic Salts (g/liter) | Amino Acids (g/liter) | Vitamins (g/liter) | Other (g/liter) |
|---|---|---|---|
| CaCl2 (anhydrous) | L-Alanine 0.00445 | D-Biotin 0.00000365 | D-Glucose 3.15100 |
| 0.11665 | L-Arginine•HCl 0.14750 | Choline Chloride 0.00898 | HEPES 3.57480 |
| CuSO4 (anhydrous) | L-Asparagine•H2O | Folic Acid 0.00265 | Hypoxanthine 0.00239 |
| 0.0000008 | 0.00750 | myo-Inositol 0.01261 | Linoleic Acid 0.000044 |
| Fe(NO3)3•9H2O 0.00005 | L-Aspartic Acid 0.00665 | Niacinamide 0.00202 | Phenol Red, Sodium Salt |
| FeSO4•7H2O 0.000417 | L-Cystine•HCl•H2O | D-Pantothenic Acid | 0.00810 |
| MgSO4 (anhydrous) | 0.01756 | 0.00224 | Putrescine•2HCl 0.00008 |
| 0.08495 | L-Cystine•2HCl 0.03129 | Pyridoxine•HCl 0.00203 | Pyruvic Acid•Na 0.05500 |
| KCl 0.3118 | L-Glutamic Acid 0.00735 | Riboflavin 0.00022 | DL-Thioctic Acid |
| NaHCO3 1.20000 | L-Glutamine 0.36510 | Thiamine•HCl 0.00217 | 0.000105 |
| NaCl 7.00000 | Glycine 0.01875 | Vitamin B-12 0.00068 | Thymidine 0.000365 |
| Na2HPO4 (anhydrous) | L-Histidine•HCl•H2O | | |
| 0.07100 | 0.03148 | | |
| NaH2PO4•H2O 0.06250 | L-Isoleucine 0.05437 | | |
| ZnSO4•7H2O 0.000432 | L-Leucine 0.05895 | | |
| | L-Lysine•HCl 0.09135 | | |
| | L-Methionine 0.01724 | | |
| | L-Phenylalanine 0.03548 | | |
| | L-Proline 0.01725 | | |
| | L-Serine 0.02625 | | |
| | L-Threonine 0.05355 | | |
| | L-Tryptophan 0.00902 | | |
| | L-Tyrosine•2Na•2H2O | | |
| | 0.05582 | | |
| | L-Valine 0.05285 | | |

To fully exploit the potential of pluripotent cells for drug discovery, testing, and transplantation therapy, derivation and growth of these cells under fully-defined and, ideally, xeno-free, conditions is desirable.

WO/2012/019122, incorporated by reference in its entirety herein, describes E8 medium, a fully-defined medium for pluripotent cells. E8 medium is a chemically defined stem cell medium that is the most widely published feeder-free cell culture medium for human ES cells and iPS cells, with established protocols for applications ranging from derivation to differentiation. WO/2012/019122 and Chen et al. (Nature Methods. 2011 Apr. 10; 8(4):424-429, incorporated by reference in its entirety herein), describe the E8 medium. E8 medium is completely defined by eight components-DMEM/F12, L-ascorbic acid, selenium, transferrin, NaCHO3, Insulin, FGF2, TGF-beta or nodal.

E8 medium is not able to support cell growth at higher density, and it is difficult to maintain or differentiate cells at confluent density. Among the phenotypes related to high density culture with E8, are that most cells died after 1-2 days of over-confluent culture, and the surviving cells are often abnormal.

High density cell culture is important for a number of reasons, including consistency of maintenance, to avoid the arise of abnormal stem cells, stem cells need high density to properly differentiate, and most current protocols use high density culture. Previous attempts to grow cells at high density have included the following supplements to the culture medium: small molecules (ROCK inhibitors, Caspase inhibitors), lipid carriers (different cyclodextrins), albumin peptides, polymers (Polyvinylpyrrolidone, Poly(vinyl alcohol)), growth factors (different bioactive proteins), nutrients (vitamins, energy source), albumin, starches, alginate, oxygen level, CDM, Chemically defined medium for high density cell culture. None of these approaches were successful.

The present invention takes a new direction, and features a novel medium for the culture of stem cells. The stem cell can be a totipotent, pluripotent, multipotent, oligopotent or unipotent stem cell. The stem cell can be an embryonic stem cell, an induced pluripotent stem cell, a fetal stem cell or an adult stem cell. In certain embodiments, the stem cell is a mammalian stem cell, for example a human stem cell, and more particularly a human embryonic stem cell or a human induced pluripotent stem cell.

The present invention is based, in part, on the surprising finding that addition of certain factors to low protein culture medium enhances pluripotent stem cell survival and differentiation efficiency.

Low protein medium refers to a medium that has a low percentage of protein. For example, a “low protein medium” can contain less than about 10%, 9%, 8%, 7%, 6%, 5%, 4%, 3%, 2% or 1% serum, wherein the culturing capacity of the medium is still observed. In certain exemplary embodiments, the low protein medium is an albumin free medium. In further exemplary embodiments, the low protein medium is E8 medium, as described herein.

Factors that have been added to the low protein medium to support pluripotent stem cell survival and differentiation include a combination of factors, as set forth below. The factors can be membrane stabilizer or volume expander, optionally, but not limited to, trehalose, CM-Dextran, polysucrose and glycogen.

Factors can be added for growth factor control, for example, but not limited to heparin and heparan sulfate.

Factors can be added to supply cellular membrane components and essential lipids, and optionally are a lipid mix. In certain embodiments, the lipid mix comprises arachadonic acid, cholesterol, DL-alpha-tocopherol acetate, ethyl alcohol 100%, linoleic Acid, linolenic Acid, myristic acid, oleic acid, palmitic acid, palmitoleic acid, Pluronic F-68, stearic acid, tween-80. In certain exemplary embodiments, the lipid mix comprises arachadonic acid (2 mg/ml), cholesterol (220 mg/ml), DL-alpha-tocopherol acetate (70 mg/ml), ethyl alcohol 100%, linoleic Acid (10 mg/ml), linolenic acid (10 mg/ml), myristic acid (10 mg/ml), oleic acid 10 mg/ml), palmitic acid (10 mg/ml), palmitoleic acid (10 mg/ml), Pluronic F-68 (90000 mg/ml), stearic acid (10 mg/ml), tween-80 (2200 mg/ml). In further exemplary embodiments, the lipid mix is commercially available as Chemically Defined Lipid Concentrate (Cat#11905031) from Life Technologies.

In certain embodiments, effective concentrations are as follows:

Trehalose, CM-Dextran, Glycogen, beta-cyclodextrin, beta-hydrodextrin, N-Acetyl-Glucosamine-Methyl alpha-D-Glycogyrate, Methyl beta-D-glycogyrate, Dextrin 100K, Methyl-1-beta-cyclodextrin, are optionally used at concentrations ranging from 0.5-40 mg/ml, in certain embodiments optionally 2-20 mg/ml, for example 2, 5, 10, and 20 mg/ml. In certain embodiments, Trehalose, CM-Dextran, Glycogen, Albumin, beta-cyclodextrin, beta-hydro dextrin, N-Acetyl-Glucosamine, Methyl alpha-D-Glycogyrate, Methyl beta-D-glycogyrate, Dextrin 100K, Methyl-1-beta-cyclodextrin, are optionally used at concentrations of 5 mg/ml and 10 mg/ml.

Polysucrose is optionally used at a concentration of 4-20 mg/ml, for example 5, 10, 15 and 20 mg/ml. In certain embodiments, polysucrose is used at a concentration of 10 mg/ml.

Glycogen is optionally used at a concentration of 1-20 mg/ml, for example 1.0, 2.0, 2.5, 5.0, 10 and 20 mg/ml. In certain embodiments, glycogen is used at a concentration of 2.5 mg/ml and 5 mg/ml.

Heparin is optionally used at a concentration of 0.5-20 ug/ml, for example 0.5, 1.0, 2.0, 5.0, 10, and 20 ug/ml when cells are fed at confluent density. In certain embodiments, heparin is used at a concentration of 20 ug/ml. In other embodiments, heparin is used at a concentration of 2 ug/ml when cells are fed one day before reaching confluency. Factors can also be removed from the low protein medium. For example, in certain embodiments, growth factors are removed from the medium. In particular, TGFβ, FGF and insulin are removed from the low protein medium.

In certain embodiments of the instant invention, heparin-containing or other related low protein medium described herein can be applied to cells at any of days −2, −1, 0, 1, 2, 3, 4, 5, 6, 7, 8, 9 and/or 10, with a regularity of once a day, twice a day, three times a day, four times a day, etc., e.g., hourly. Similarly, application of such low protein medium can be withdrawn or halted at any of days −1, 0, 1, 2, 3, 4, 5, 6, 7, 8, 9, 10, 11 and/or 12 or later.

Accordingly, the present invention features a medium for the culture of stem cells comprising one or more polysaccharides. In certain embodiments, the medium is a low protein medium. In certain embodiments, the low-protein medium is an albumin free medium. In further embodiments, the one or more polysaccharides is selected from the group consisting of trehalose, CM-Dextran or glycogen. In other further embodiments, the medium further comprises heparin, heparan sulfate or dextran sulfate. In still other further embodiments, the medium further comprises a lipid mixture.

Optionally, the medium can support cell culture objectives. In particular, the cell culture objectives are selected from the group consisting of cell survival, maintenance, passaging, proliferation, pluripotency, cloning, differentiation and induced pluripotent stem (iPS) cell derivation.

In another aspect, the invention features a method for deriving a cardiac cell under defined conditions, the method comprising the step of culturing a pluripotent stem cell in the low protein medium as described herein.

The expression of various markers specific to cardiomyocytes is detected by conventional biochemical or immunochemical methods. For example, an immunochemical method such as immunohistochemical staining or immunoelectrophoresis may be used. In these methods, marker-specific polyclonal antibodies or monoclonal antibodies can be used which react with cardiomyocyte progenitor cells or cardiomyocytes. Antibodies for individual specific markers are commercially available, and can be easily used. Markers specific to cardiomyocyte progenitor cells or cardiomyocytes include, but are not limited to, for example TBX5, TNNT2, NKX2-5, myosin heavy and light chains, α-actinin, troponin 1, ANP, GATA-4, MEF-2c and the like.

Alternatively, expression of cardiomyocyte progenitor cell-specific or cardiomyocyte-specific marker genes can also be confirmed by molecular biological methods, such as microarray, reverse transcriptase polymerase chain reaction (RT-PCR) and hybridization analysis, which have been commonly used in the past for amplifying, detecting and analyzing mRNA encoding any marker proteins. The nucleic acid sequences encoding marker proteins specific to cardiomyocyte progenitor cells and cardiomyocytes (such as TBX5, TNNT2 NKX2.5) are already known and are available through public databases such as GenBank, and the marker-specific sequences needed for use as primers or probes can be easily determined.

Physiological indexes can also be used additionally to confirm differentiation of ES cells into cardiomyocytes. For example, useful markers include the ability of cells derived from ES cells to beat spontaneously, and the ability of cells derived from ES cells to react to electrophysiological stimulus through various ion channels expressed on the cells.

Any method suited to inducing differentiation of cardiomyocytes can be used as the culture method for preparing cardiomyocytes from hPSC (human pluripotent stem cells, ESC/iPSC) cells in the present invention. For example, cell culture can be in adherent culture, suspension culture, soft agar culture, micro-carrier culture and the like.

Cardiomyocytes prepared according to the present invention can be used as myocardial regeneration drugs or heart disease treatment drugs. Examples of heart disease include myocardial infarction, ischemic heart disease, congestive heart failure, hypertrophic cardiomyopathy, dilative cardiomyopathy, myocarditis, chronic heart failure and the like. When used as myocardial regeneration drugs or heart disease treatment drugs, cardiomyocytes prepared according to the present invention can be included in any form as long as the purity is high, such as cells suspended in the medium or other aqueous carrier, cells embedded in a biodegradable substrate or other support, or cells made into a single-layer or multilayer myocardial sheet.

Although not particularly limited to these, methods for transporting the cardiomyocytes prepared according to the present invention to a damage site include direct injection into the heart via an open chest and a syringe, methods of transplantation via a surgical incision in the heart, and methods of transplantation via the blood vessels using a catheter, all of which have been described in the art (Murry et al., Cold Spring Harb. Symp. Quant. Biol. 67:519, 2002; Menasche, Ann Thorac. Surg. 75:S20, 2003; Dowell et al., Cardiovasc. Res. 58:336, 2003). Good therapeutic effects have been reported when cardiomyocytes collected from a fetal heart were transplanted by such methods to the hearts of animals with heart damage (Menasche, Ann. Thorac. Surg. 75:S20, 2003; Reffelmann et al., Heart Fail. Rev. 8:201, 2003). Cardiomyocytes derived from ES cells have characteristics similar to those of cardiomyocytes derived from fetal hearts (Maltsev et al., Mech. Dev. 44:41, 1993; Circ. Res. 75:233, 1994). Further, a high take rate equivalent to that achieved with fetal myocardial transplantation has been confirmed in animal experiments in which cardiomyocytes derived from ES cells were actually transplanted into adult hearts (King et al., J. Clin. Invest. 98:216, 1996). Accordingly, it is expected that supplementary transplantation of cardiomyocytes prepared according to the present invention into diseased heart tissue should stimulate improved heart functions.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Deriving Human Pluripotent Stem Cells in E8 Based Growth Conditions

Human pluripotent stem cells (hPSCs), including human embryonic stem cells (hESCs) and human induced pluripotent stem cells (hiPSCs), have the potential to become the source materials for cell-based therapy, so the quality of the stem cells has great impact on how the cells could be utilized in future applications. Derivation and maintenance conditions have critical role determining the iPSC quality, mainly due to the involvement of animal products and feeder cells. A procedure was developed to derive and maintain hiPSCs in chemically defined media.

The following sets forth how to derive hiPSCs in E8 based growth conditions. This method has been successfully used on fibroblast, pre-adipocyte and HUVEC. The procedure can be adapted to most common reprogramming methods, such as Lentivirus, Episomal DNA and Sendai Virus. In comparison with E8 medium and vitronectin (or synthetic surface), human pluripotent stem cells could be maintained in an enzyme-free, xeno-fee and chemically defined environment. To simplify the description, Sendai Virus is used to reprogram fibroblasts, and matrigel is used as the coating surface for cell culture in this protocol. However, the invention is not limited to either.

Plate Coating.

First (1), pour cold 12 ml of DMEM/F12 in conical tube, and use 1.5 ml to re-suspend 2 mg frozen matrigel with 5 ml pipet. Matrigel should be in freezer right before the experiment. Next, (2) rinse the matrigel tube again with the same media. (3) Mix the matrigel well, plate 1 ml in each well of 6-well), and shake well to cover all the surface. (4) Leave the plate at room temperature or 37° C. for at least 30 minutes. Or coat overnight at 4° C.

EDTA Dissociation Buffer: For iPSCs

(1) Add 500 ul 0.5M EDTA and 0.9 g NaCl into 500 ml Calcium/Magnesium free PBS (Invitrogen #14190). (2) Sterilize by filtration, and store at 4° C.

Reprogramming with. Sendai Virus.

1—Day 0—Take low passage # fibroblast culture and plate in 1 well of a 6 well dish so that cells will be about 80% confluent next day (100K or 150K cells/well) in fibroblast Media (usually 10% FBS and Pen/Strep, L-glut, NEAA in DMEM).
2—Day 1—Thaw the four Sendai Viruses on ice, mix together, and drop-wisely add them to the cells. Incubate at 37° C.
3—Day 2—If cells look good in the morning and nearly confluent, prepare plates for splitting. Coat 6 well plates with Matrigel.
4—Still Day 2—Pass reprogramming well using TryPLE (Note, some lines do not come off plate well with TryPLE, with those lines, it may be advantageous to wash plate 2× with EDTA before adding TryPLE). Incubate 5 min in incubator, then wash off plate and dilute with fibroblast media.
5—Spin cells down and resuspend in fibroblast media. Plate on Matrigel (1 well of infected cells into 2×6 well plates coated with matrigel), also in Reprogramming Medium I.
6—Keep cells in Reprogramming Medium I, feeding them every other day for 3-5 days.
7—Day 5 or 7 (approximately)—change media to Reprogramming Medium II, and 100 RIVI Sodium Butyrate can be added to improve reprogramming efficiency.
8—Continue feeding every other day.
9—Depending on cell density and the appearance of any iPS colonies in the reprogramming plates, cells may need to be passed with EDTA at some point in the next 2 weeks. Also, it may be necessary to start feeding E8 medium without sodium butyrate.
10—Around Day 20-25, colonies should be ready to try picking. Around this time original reprogramming plates should begin being fed normal E8 media (with TGFβ1) daily, if they haven't been switched to this already.
11—For picking-prep a 24 well plate by coating with Matrigel. After coating, add E8 (TGFβ1 media) with 1× Rock inhibitor added to each well. Spray microscope and surrounding area as well as pipet and box of tips with alcohol. Wear face mask. Find colonies under scope with 4× objective. Using P20 with tip, circle around colony until it is loosened from surrounding cells. Also using tips, cross hatch the colony so it will come off plate in smaller pieces. Then use pipet to push colony off plate and suck it into pipette. Transfer colony pieces into 1 well on the 24 well plate. Repeat with other colonies. The day after picking, change media to E8 (TGFb1) without rock inhibitor. Feed daily until colony is big enough to pass.
12—When colony is ready to pass, use EDTA to pass and leave some of the cells in the original well of the 24 well while transferring most to a new Matrigel coated well on a 12 or 6 well plate. (in Rock inhibitor).
13—When expanding each colony, keep track of passage number (picking into 24 well is passage 1). Once you have a well in a 6 well ready to pass, freeze 2 vials down and leave some cells in the well to continue to grow them.
14—The usual order for cells is—expand and freeze 2 vials from first well. Expand well on next passage to 2-3 wells. Freeze 4-6 vials when ready, and continue growing cells. Freeze at least 6 vial, up to 10 for each clone. During one passage, pass a small amount onto 12 well plate for APS staining. After getting enough cells for freezing, continue growing to harvest some for FACS staining.

### Materials

### Cell Materials:

### Human Fibroblasts

Human iPSCs

### Cell Culture Media:

Fibroblast medium: 10% Fetal Bovine Serum in DMEM, lx Non-essential amino acid.
Basic chemically defined reprogramming medium I:
DMEM/F12, 64 mg/L L-Ascorbic acid 2-phosphate magnesium salt, 14 pg/L Sodium Selenite, 10.7 mg/L Holo-transferrin 10.7 mg/L, 100 μg/L basic FGF, 20 mg/L Insulin and 1 pM Hydrocortisone. Adjust to pH7.4 with 340 mOsm osmolality.
Basic chemically defined reprogramming medium II:
DMEM/F12, 64 mg/L L-Ascorbic acid 2-phosphate magnesium salt, 14 μg/L Sodium Selenite, 10.7 mg/L Holo-transferrin 10.7 mg/L, 100 μg/L basic FGF, and 20 mg/L Insulin. Adjust to pH7.4 with 340 mOsm osmolality. 100 uM Sodium Butyrate can be added to improve reprogramming efficiency.
Chemically defined human ESC/iPSC E8 medium:
DMEM/F12, 64 mg/L L-Ascorbic acid 2-phosphate magnesium salt, 14 μg/L Sodium Selenite, 10.7 mg/L Holo-transferrin 10.7 mg/L, 100 pg/L basic FGF, 1.8 pg/L TGFB1, 20 mg/L Insulin. Adjust to pH7.4 with 340 mOsm osmolality.

### EDTA PBS (1000 ml)

| Ingredient | Amount | | Company | Catalog# |
|---|---|---|---|---|
| PBS | 500 | ml | Life Technology | 14190-250 |
| 0.5M EDTA | 0.5 | ml | K.D. Biomedical | RGF3130 |
| NaCI | 0.9 | g | Sigma | 5886 |

### Medium Reagents

| Ingredient | Company | Catalog# |
|---|---|---|
| DMEM/F12 | Life Technology | 11330 |
| L-Ascorbic acid 2-phosphate | Sigma | A8960 |
| magnesium salt | | |
| Sodium Selenite | Sigma | S5261 |
| Sodium Chloride | Sigma | S5886 |
| Holotransferrin | Sigma | T0665 |
| Basic FGF | Peprotech | 100-18B |
| TGFB1 | R&D Systems | 240-B/CF |
| Insulin | Sigma | 19278 |
| Hydrocortisone | Sigma | H0396 |
| Sodium Butyrate | Sigma | B5587 |

### Materials

Inverted microscope (i.e., Nikon TE or Olympus IX or Zeiss Promo Vert)
Biosafety cabinet for cell culture
CO2 incubator with controlling and monitoring system for CO2, humidity
and temperature
Cell culture disposables: Tissue culture dishes, centrifuge tubes, pipettes,
pipette tips, cell strainer etc.

Troubleshooting for the below issues is described, although not limited to the following:
Xeno-Free Condition for iPSC Derivation

Matrigel could be replaced with VitronectiP recombinant protein.

PBS-Containing fibroblast medium could be replaced with defined fibroblast medium.

### Low Reprogramming Efficiency

Use sodium butyrate to improve the efficiency.

### Initial Fibroblasts are not Actively Proliferating or at High Passage.

Increase the experiment scale, and use more starting cells and viruses.

### Example 2

### Identification of New Factors that Enhance Pluripotent Stem Cell Survival and Differentiation

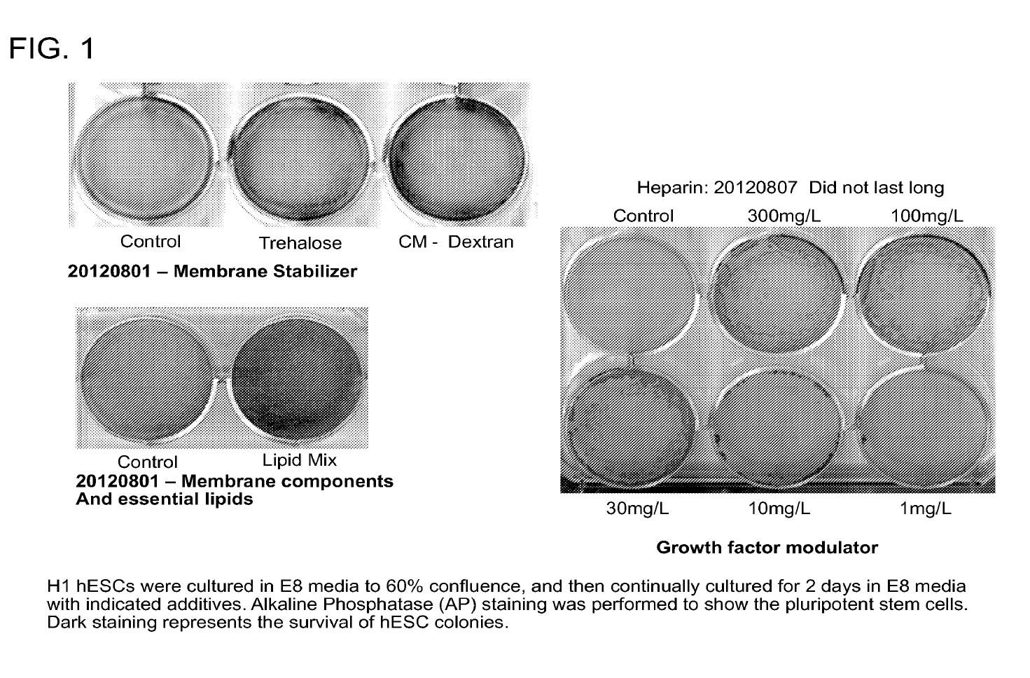

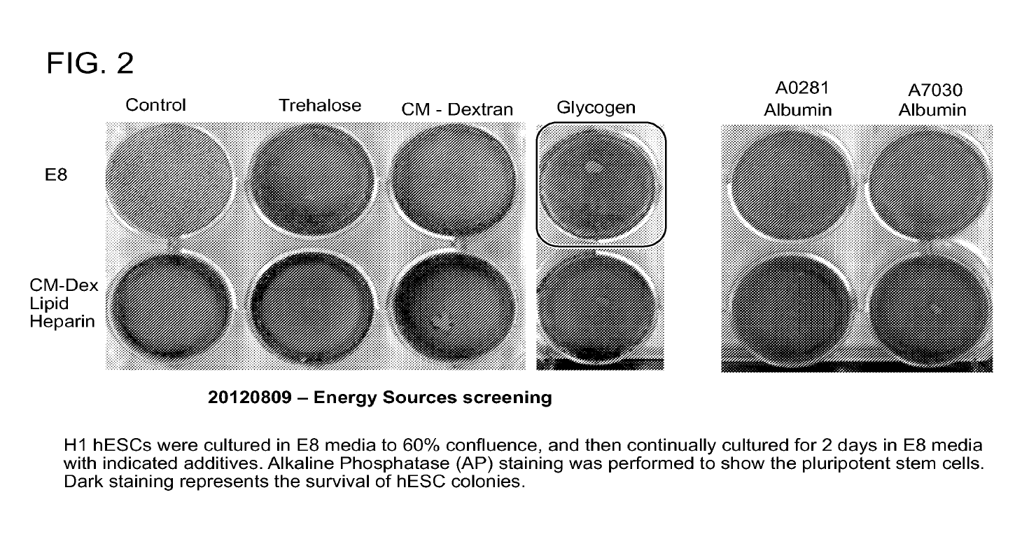

First, initial screening assays were carried out with different additives. In this assay, human embryonic stem cells (ESCs) are grown to near confluence in E8 medium. Culture is continued with different additives as indicated in FIGS. 1 and 2. The additives in the set of experiments described herein are selected from trehalose, CM-Dextran, lipid mix, heparin, glycogen and albumin Cells that were untreated served as the controls. The protocol is set forth below:
1. Human ESCs were expanded with EDTA dissociation at 1:6 ratio.
2. E8 medium was changed every day.
3. After 2-3 days, when cell density reached around 70-80% confluency.
4. Change medium, and different treatments were applied to the cells in addition to fresh E8 every day.
5. Cells in E8 started to die 1-2 days after they reached 100% confluency.
6. Plates were stained with Alkaline Phosphatase staining (APS) kit (Vector Lab) to determine pluripotency maintenance. After a few days, the plates are stained with Alkaline Phosphatase Staining (APS). The cell viability reagent PrestoBlue can also be used to measure live cells. In this protocol
1. Human ES cells were cultured at 50% confluence in E8 media when polymers were added into the media.
2. After another two days of culturing, the cell viability reagent PrestoBlue (Life Technologies) was added into the media and incubated for one hour.
3. Cell viability was detected by fluorescence using 560 nm excitation and 590 emission.

Results from these initial screening assays are shown in FIGS. 1 and 2.

The results of these experiments showed that new factors, in particular, trehalose, glycogen, CM-Dextran and heparin, enhanced pluripotent stem cell survival and differentiation efficiency.

Trehalose (0.2-1%), shown below, is a natural alpha-linked disaccharide formed by an α,α-1,1-glucoside bond between two α-glucose units.

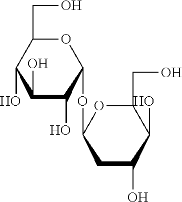

Glycogen (0.1-1%; also called dextrin in older texts, which is not to be confused with the polysaccharide called dextran that is made by bacteria), shown below, is found principally in muscle and liver cells, where it serves as a readily accessible depot for the storage of glucose.

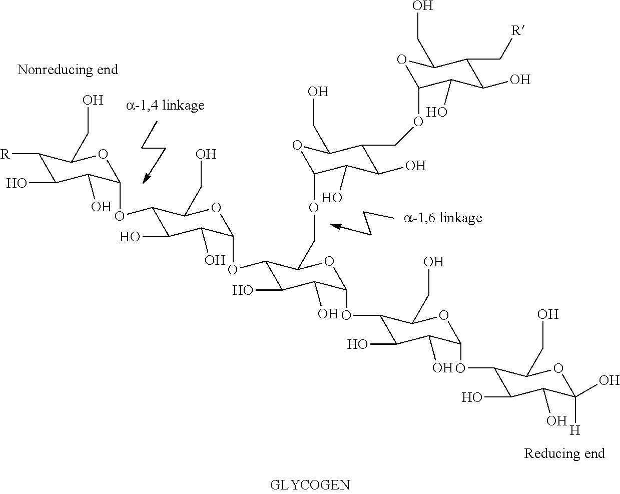

CM-dextran (0.2-1%), shown below, consists of a dextran backbone substituted with carboxymethyl substituents imparting a polyanionic character to the product. The carboxymethyl content corresponds to about 1 CM group for every 5 glucose units.

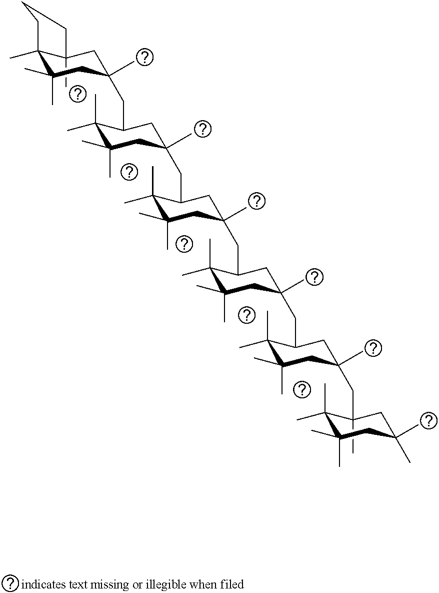

Heparin (20 mg/L), shown below, also known as unfractionated heparin, a highly sulfated glycosaminoglycan, is widely used as an injectable anticoagulant, and has the highest negative charge density of any known biological molecule.

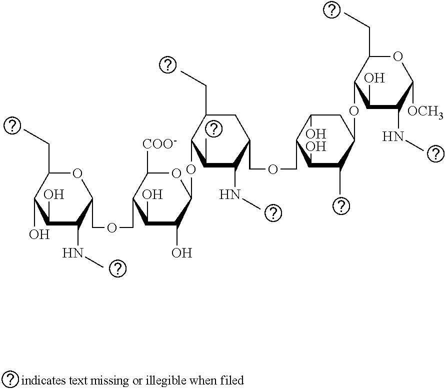

Heparan sulfate is a linear polysaccharide found in all animal tissues. It occurs as a proteoglycan (HSPG) in which two or three HS chains are attached in close proximity to cell surface or extracellular matrix proteins.

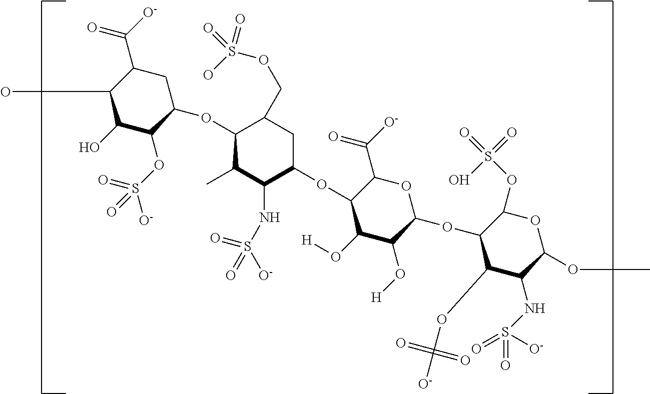

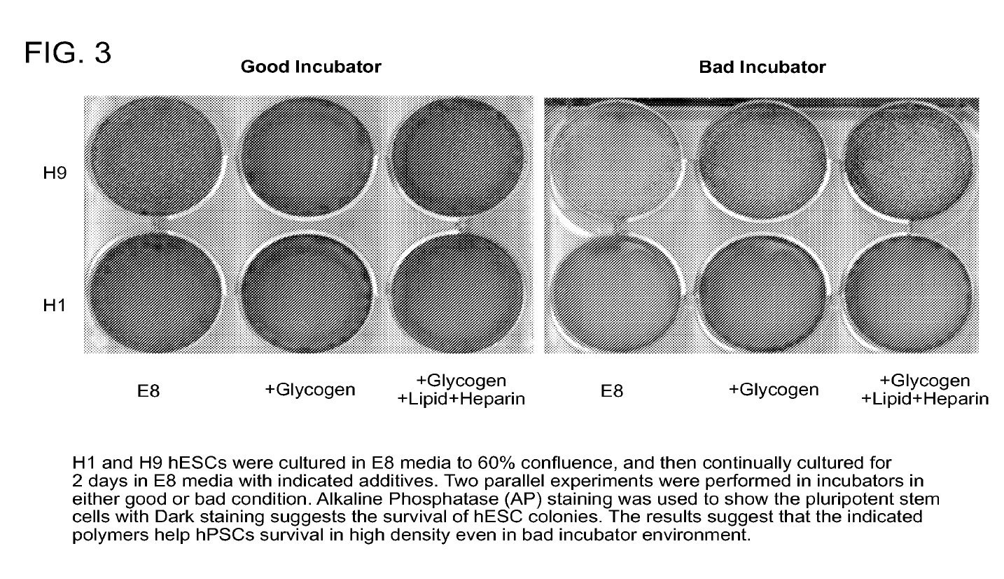

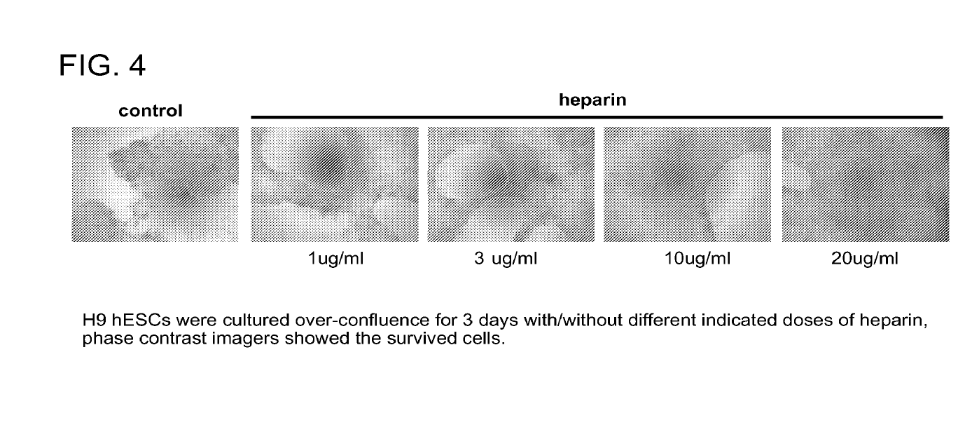

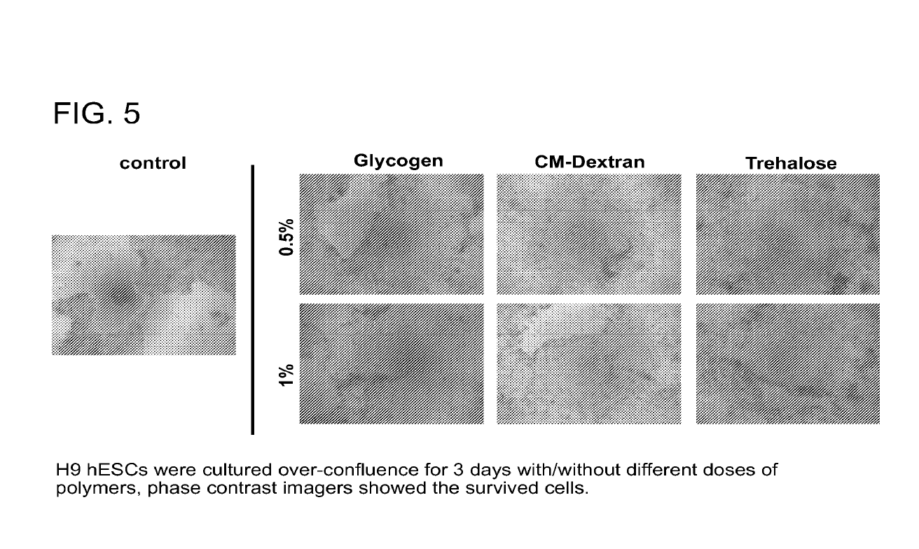

These new factors (e.g. one or more of trehalose, CM-Dextran, lipid mix, heparin, glycogen and) were also beneficial for cell maintenance in suboptimal conditions. As shown in FIG. 3, most cells do grow, albeit slower, and the cells also differentiated even in suboptimal conditions “bad incubator.” The dosages of these factors were also tested for hPSCs survival as shown in FIGS. 4 and 5.

Next, experiments were carried out with cells grown in E8 medium supplemented with one or more polysaccharides (e.g. trehalose, CM-Dextran or glycogen).

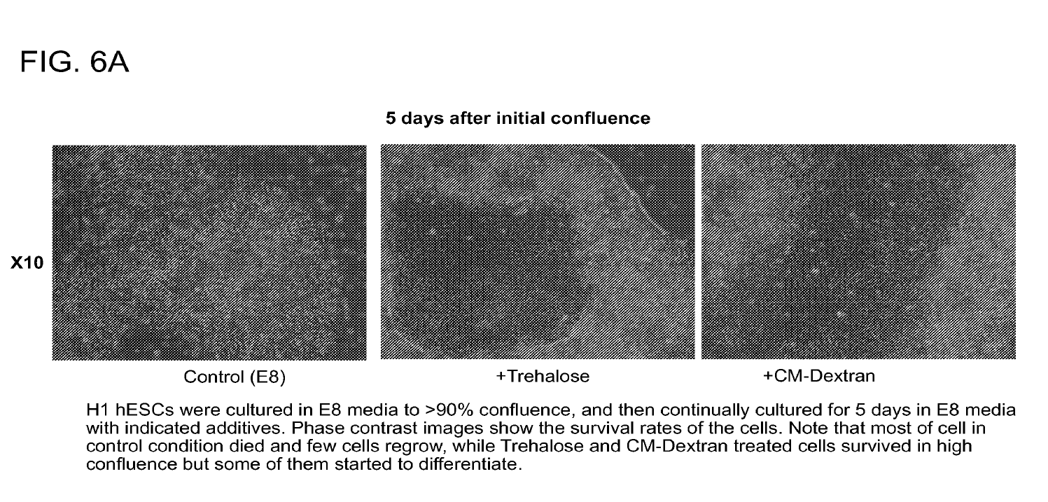

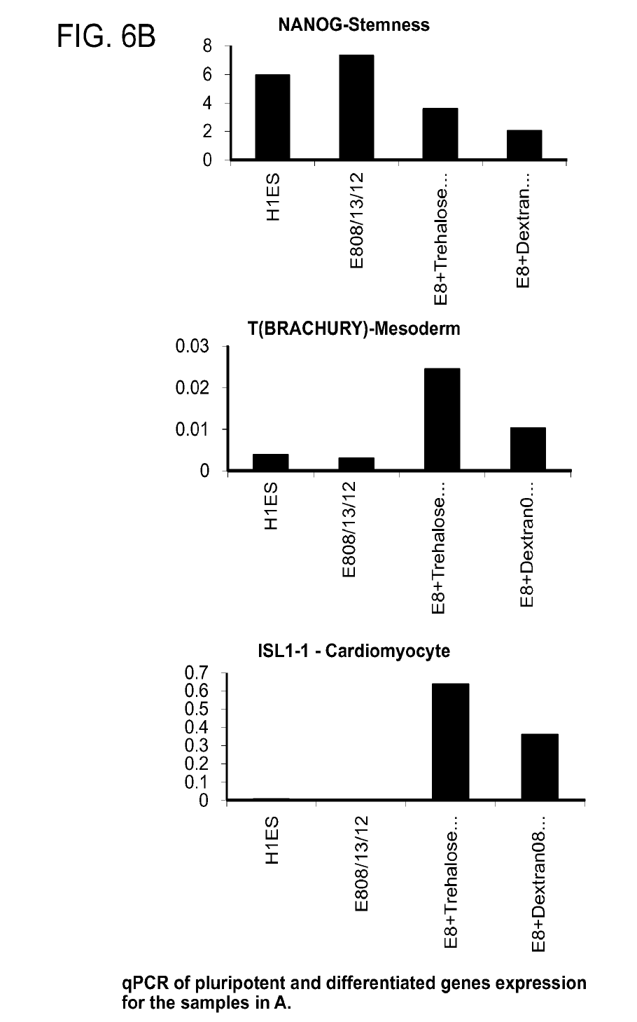

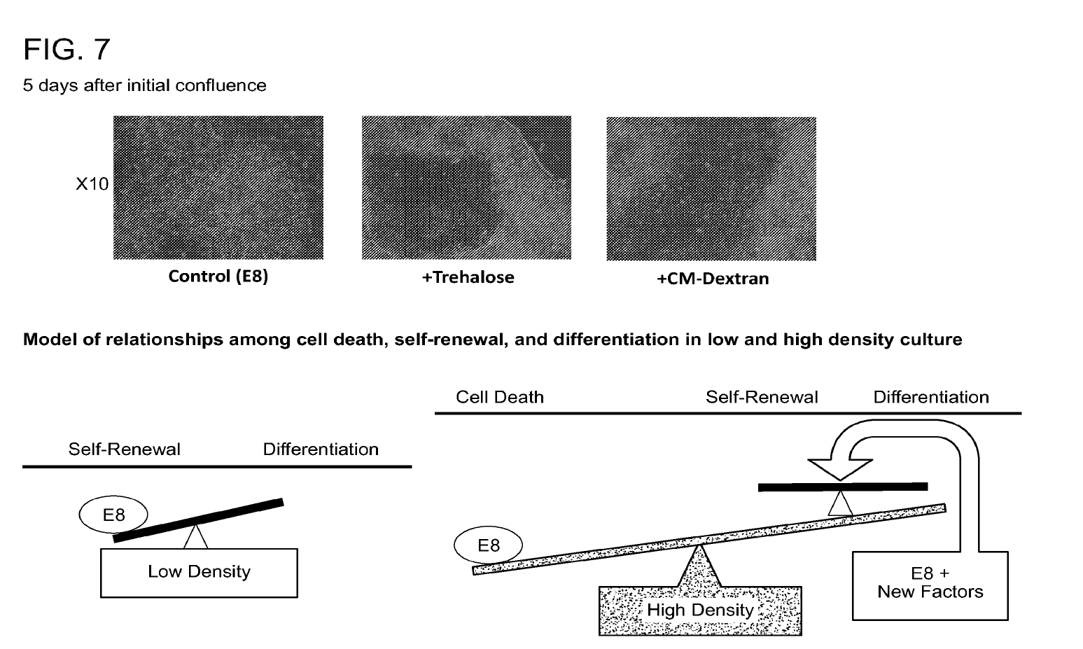

FIG. 6 shows cell proliferation and spontaneous differentiation at high density with trehalose and CM-dextran. ES cells were cultured in E8 medium supplemented with trehalose or CM-dextran. FIG. 6 shows the cells 5 days after initial confluence. As shown in the control panel, on the left, most cells died and leftover cells grew up again and did not differentiate. However, in the trehalose and CM-dextran supplemented medium, shown in the center and right panels respectively, there was little cell death, and the cells differentiated. The graphs at the bottom of FIG. 6 show that in cells cultured in E8 medium supplemented with trehalose or CM-dextran, markers of differentiation were expressed. All the data suggest that E8 maintains self-renewal and pluripotency in low-density culture. However, in high density, E8 needed supplementation of other factors which help inhibit cell death as well as balance self-renewal and differentiation (FIG. 7).

Most current differentiation protocols use undefined conditions and require high density culture. Cells cultured in E8 medium usually cannot differentiate. Therefore, the problems to be addressed in developing an E8 based differentiation platform were to decrease cell death at high density during differentiation and the difference in growth factor treatment and timing. Nine initial differentiation tests were performed—four with cardiomyoctes, one neural, one hepatocyte, one endothelial, one smooth muscle and one blood lineage.

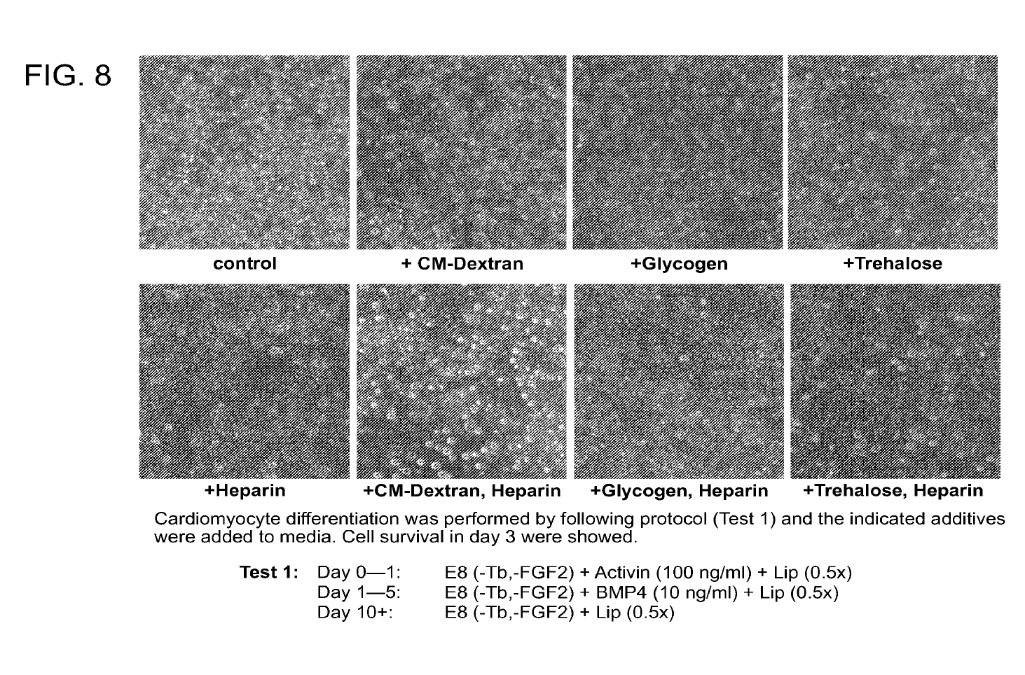

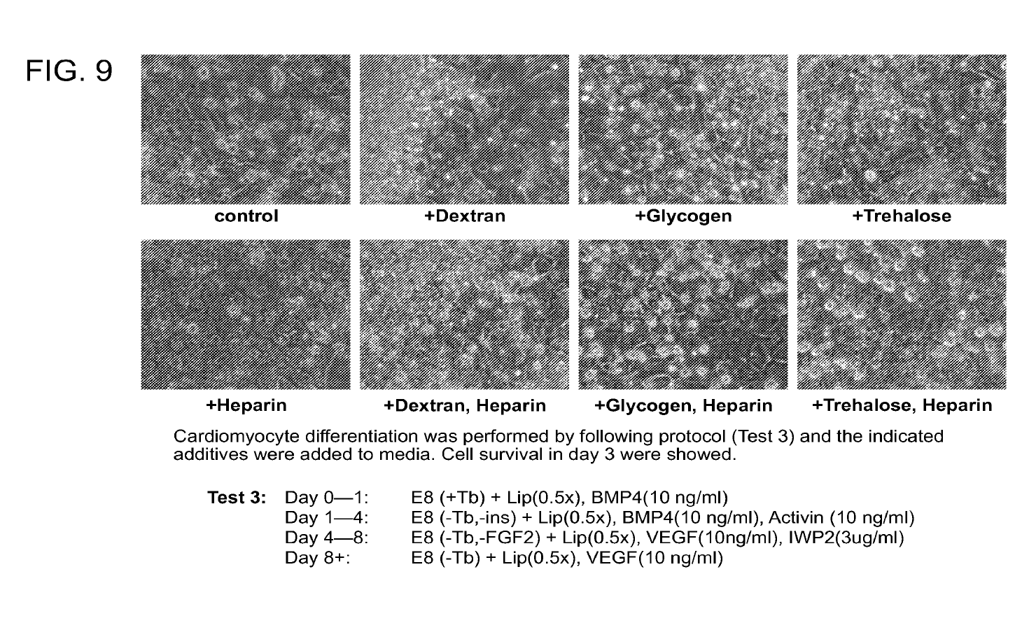

Experiments were carried out to determine if these newly identified factors suppressed cell death during differentiation. FIGS. 8 and 9 show that E8 medium supplemented with the new factors (dextran, glycogen, trehalose, heparin, dextran and heparin, glycogen and heparin, trehalose and heparin) suppressed cell death during differentiation. FIG. 8 shows cardiomyocyte differentiation that was performed by test condition 1 (Test 1), as indicated in the FIG. 8 legend. The indicated additives (CM-Dextran, Glycogen, Trehalose, Heparin, or various combinations were added to the media. Cell survival at day 3 is shown. FIG. 9 shows cardiomyocyte differentiation that was performed by test condition 3 (Test 3), as indicated in the FIG. 9 legend. The indicated additives (CM-Dextran, Glycogen, Trehalose, Heparin, or various combinations were added to the media. Cell survival at day 3 is shown. Cell density is increased in cells cultured in E8 medium supplemented with the new factors (glycogen, trehalose, heparin).

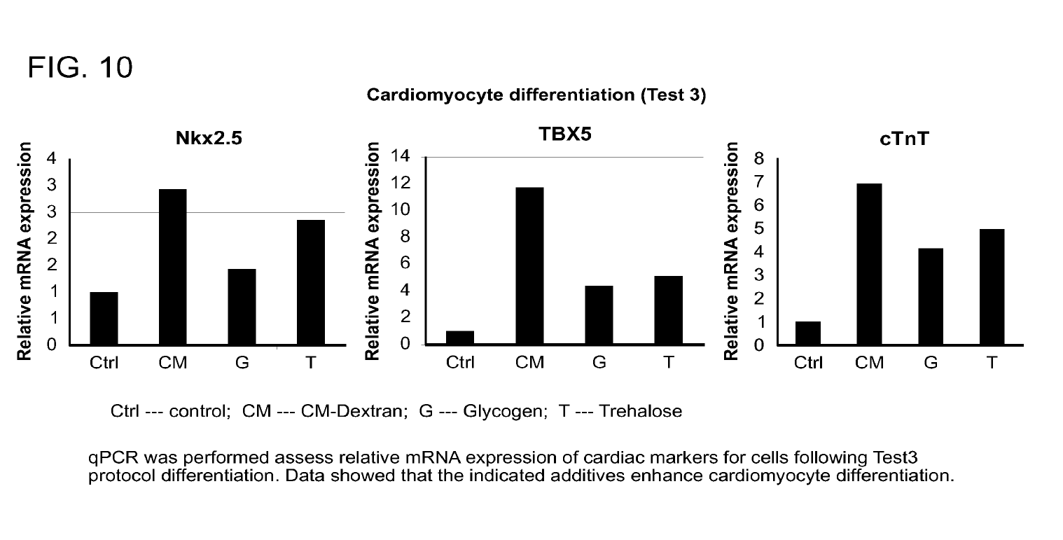

Gene expression during differentiation was examined, and it was shown that the new factors affected specific gene expression during differentiation. FIG. 10 shows the results of qPCR that was performed to assess relative mRNA expression of cardiac markers for cells following the Test 3 protocol, as described infra. FIG. 10 shows that the new factors (CM-Dextran (DM), glycogen (G), trehalose (T), promoted cardiomyocyte differentiation, as shown by mRNA expression of NKX2-5, TBX5 and TNNT2, which are markers of cardiomyocyte differentiation.

### Example 3

### Heparin Promotes Cardiac Differentiation in Chemically Defined Conditions

Cardiomyocytes are thought to be terminally differentiated. Although a small percentage of the cells may have proliferative capacity, it is not sufficient to replace injured or dead cardiomyocytes. Death of cardiomyocytes occurs, for example, when a coronary vessel is occluded by a thrombus and the surrounding cardiomyocytes cannot be supplied with necessary energy sources from other coronary vessels. Loss of functional cardiomyocytes may lead to chronic heart failure. A potential route for restoring “normal” heart function is replacement of injured or dead cardiomyocytes by new functional cardiomyocytes. It would be advantageous to transplant healthy cardiac cells from the patient's iPSCs.

At the same time, heart is often the main target of toxicity of drugs. Therefore, high quality cardiac cells are essential for excluding high-risk drugs. Patient specific cardiac cells could also be used to test drug dose for personalized therapies. It is important to generate large quantities of patient specific cardiac cells that could be used in therapy, diagnosis and screening. Human pluripotent stem cells (hPSCs) offer the potential to generate large numbers of functional cardiomyocytes from clonal and patient-specific cell sources.

Described herein is a new approach using heparin and heparan sulfate that could significantly improve the production of cardiac differentiation from human ESC and iPSCs. These experiments demonstrate that heparin can contribute to cardiac differentiation and that a new cardiac differentiation culture improves the efficiency of cardiomyocyte differentiation. It is expected that these culture conditions will be applicable to all hES cell lines and hiPSCs in general. Furthermore, the fact that these differentiation conditions are established without fetal calf serum, and thus without the presence of animal pathogens, increases the chance that these hES-derived cardiomyocytes are suitable for cardiomyocyte transplantation in patients with heart disease.

Heparin and Heparan Sulfate regulate growth factor activities, for example FGF, WNT, BMP. Further, defects in heparan sulfate synthesis lead to deficiency in cardiac differentiation. Other protocols described in the art use undefined factors and have inconsistent production depending on batch quality of source materials. For example, B27 medium is a complex medium that has shown to have quality issues and inconsistencies, for example inconsistent albumin concentrations.

The present invention provides a simple and well-defined system to produce large quantities of human cardiac cells from iPSC/ESCs.

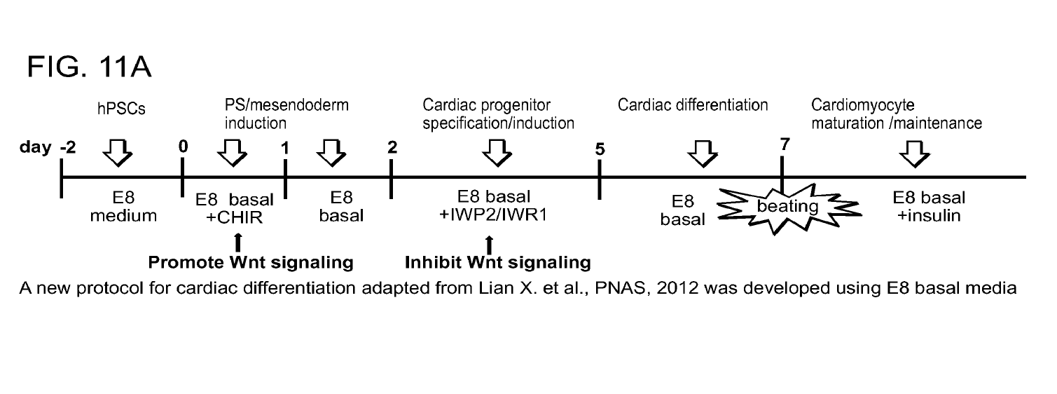

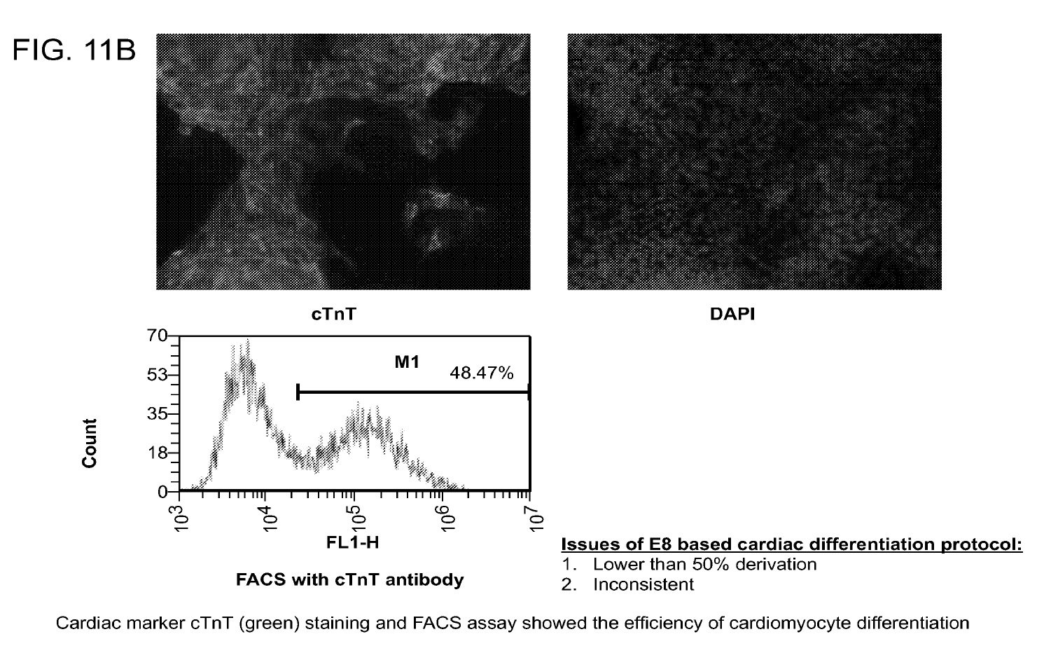

FIG. 11 shows E8 based conditions for cardiac production. As shown in FIG. 11, hPSCs were cultured in E8 medium. CHIR (CHIR99021, a GSK-3α/β inhibitor) was added to the E8 basal medium to promote WNT signaling and primitive streak (PS)/mesoderm induction (days 0-1). Next, IWP2/IWR1 was added to E8 basal medium to inhibit WNT signaling and promote cardiac progenitor specification/induction (days 2-5). The medium was changed to E8 basal medium (days 5-7) to promote cardiac differentiation. At day 7, beating of the cardiomyocytes could be detected. Cardiac maturation/maintenance was carried out in E8 medium supplemented with insulin. However, this E8 based cardiac differentiation protocol yielded a lower than 50% derivation, and was inconsistent.

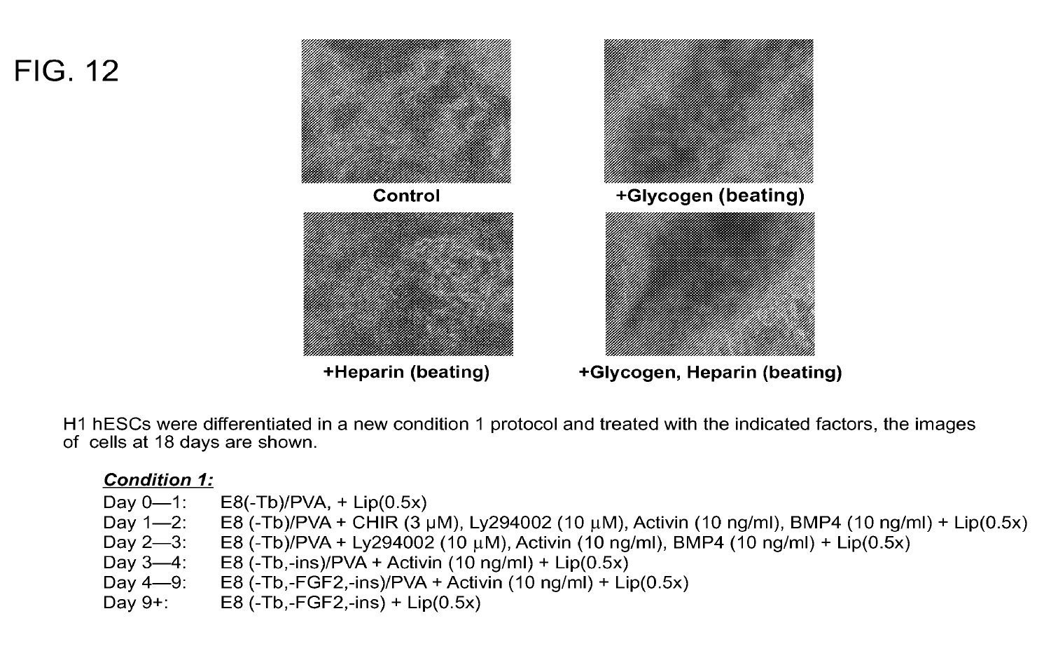

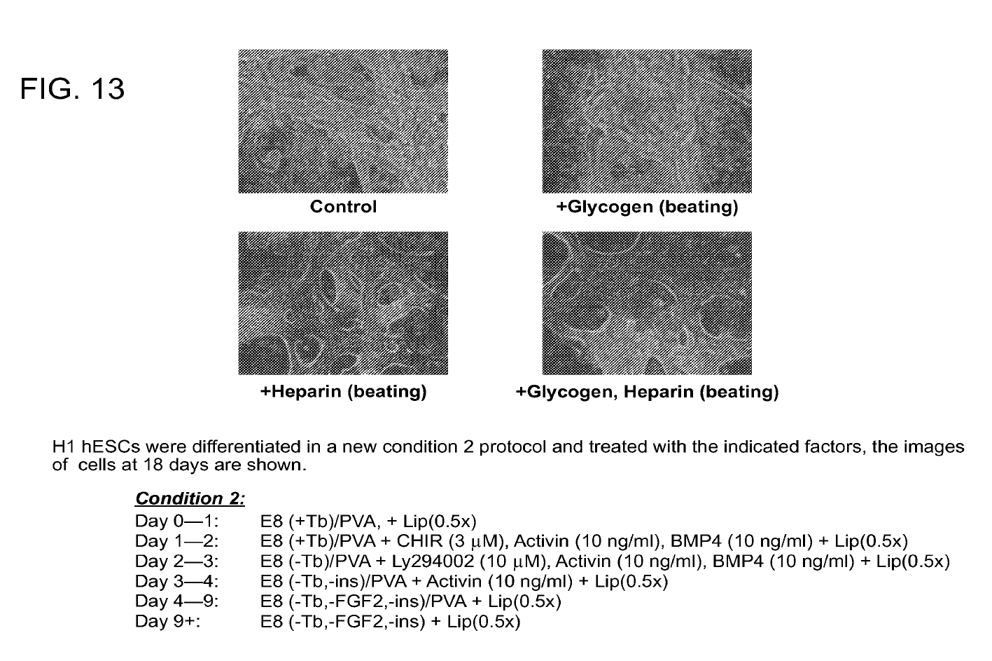

FIGS. 12 and 13 show that the new factors (Glycogen and Heparin) considerably improved cardiomyocyte differentiation in two different conditions, with/without LY294002 inhibitor. The beating cardiomyocyte foci were imaged by microscopy and video camera.

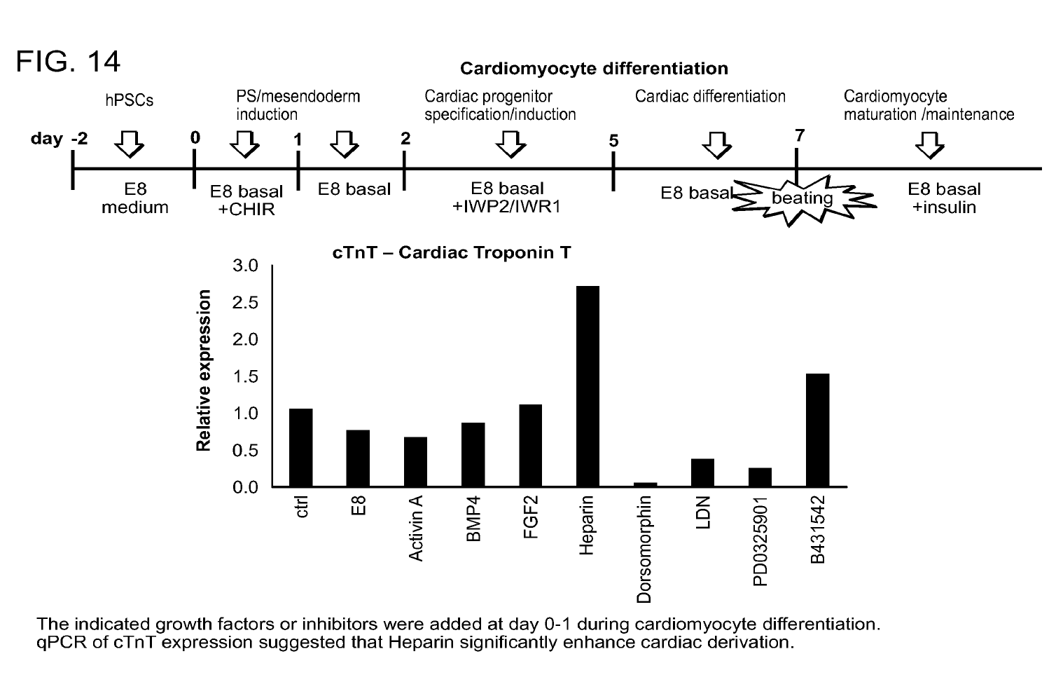

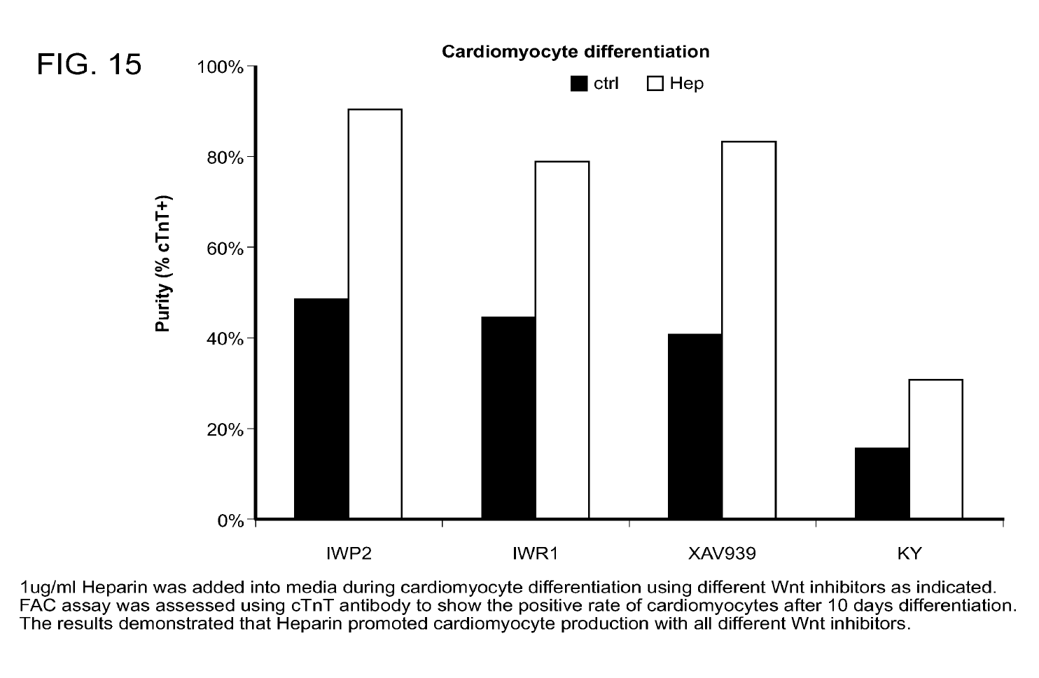

Next, screening for factors promoting cardiac differentiation was performed. As shown in FIG. 14, growth factors or inhibitors were added at day 0-1 with CHIR treatment during differentiation. mRNA from 10 days differentiated cells was collected for qPCR of cardiac Troponin T(cTnT). The results are shown in the graph in FIG. 14. When Heparin was added, relative expression of cTnT was considerably increased (similar results were observed for heparin sulfate). Heparin was also shown to be beneficial in combination with different WNT modulators. FIG. 15 shows fluorescence-activated cell sorting (FACS) with cTnT of cells that were differentiated for 10 days with different WNT inhibitors (IWP2, IWR1, XAV939, KY). As shown in FIG. 15, heparin treated cells promoted cardiac differentiation, as seen about 90% cTnT positive cells determined by FACS. This treatment can also be applied to cells in suspension culture and heparin and heparin sulfate would be effective at promoting differentiation even in suspension cultures.

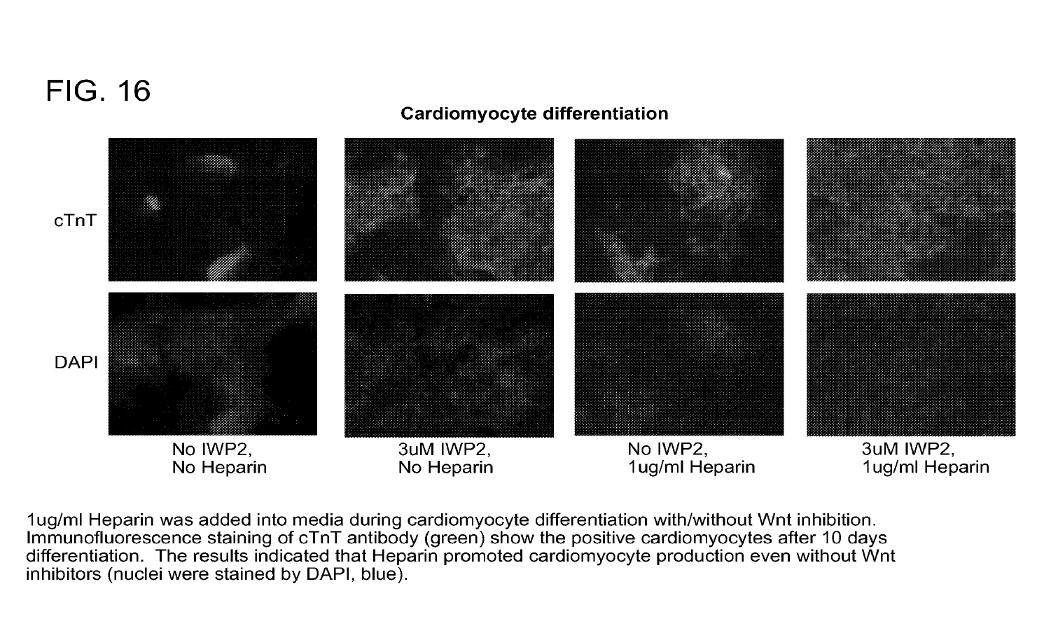

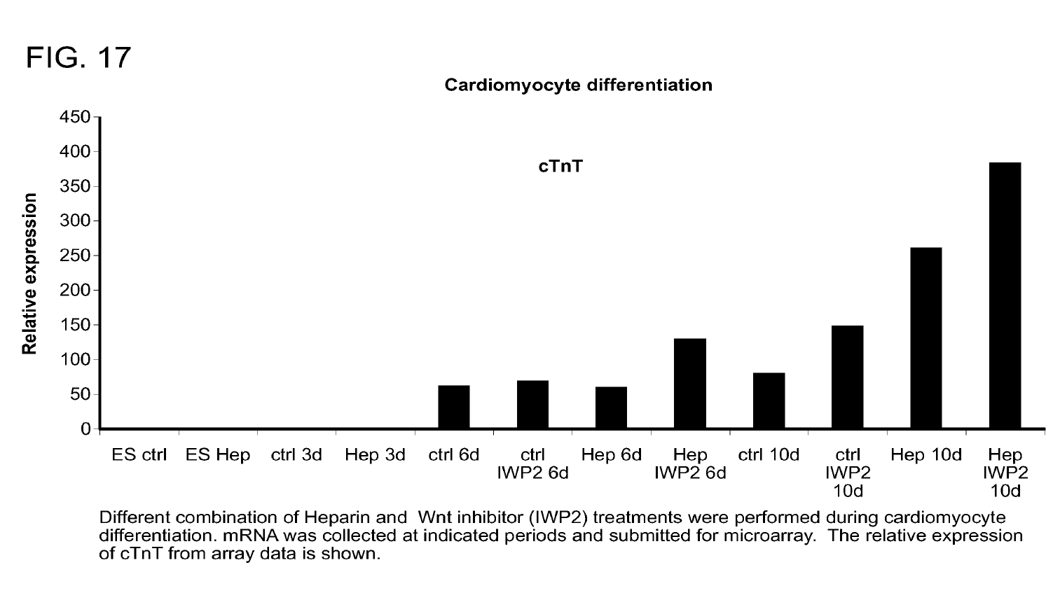

As shown in FIG. 16, it was found that heparin promotes cardiac differentiation in the absence of WNT inhibition. The panel on the left shows control, untreated cells (no IWP2, no heparin). The other panels show cells treated with 3 uM IWP2 and no heparin, no IWP2 and 1 ug/ml heparin, and 3 uM IWP2 and 1 ug/ml heparin. As shown in FIG. 16 with cTnT staining, cardiac differentiation was seen without IPW2 and in the presence of 1 ug/ml heparin. FIG. 17 shows the results of microarray experiments demonstrating heparin induced cardiac differentiation (shown by cTnT expression) independent of WNT inhibitors.

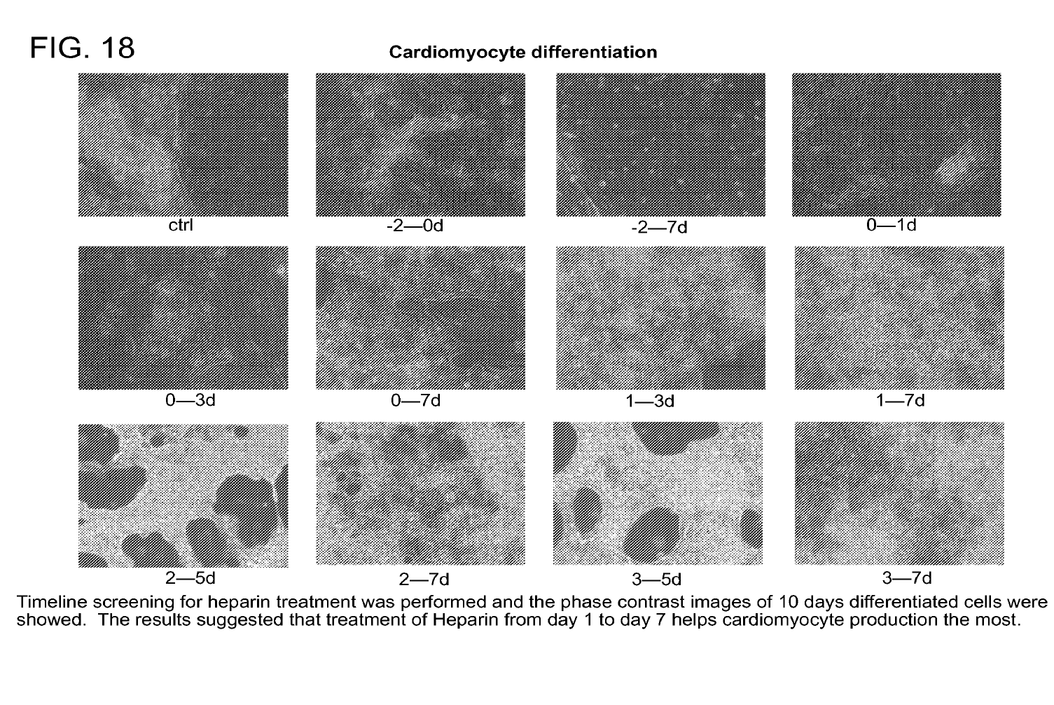

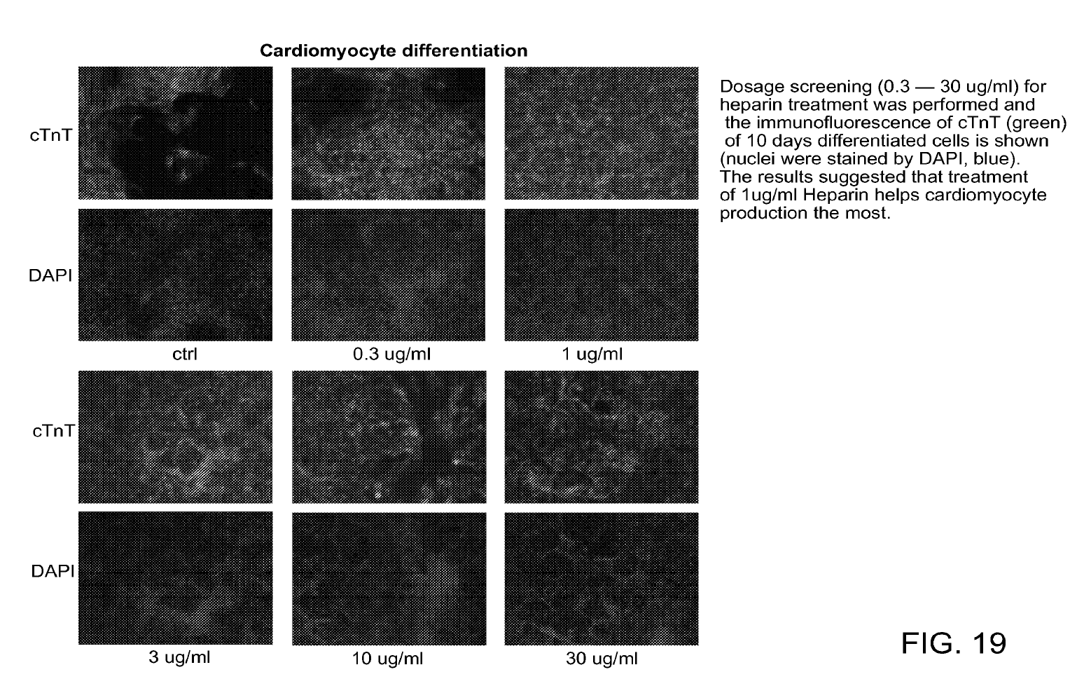

It was determined through these experiments that the timing of heparin administration affected the extent of cardiomyocyte differentiation. As shown in FIG. 18, cells were treated with 1 ug/ml at different time periods during cardiomyocyte differentiation (−2 −0 d; −2 −7 d; 0-1 d; 0-3 d; 0 −7 d; 1-3 d; 1-7 d; 2-5 d; 2-7 d; 3-5 d; 3-7 d). The results indicated that heparin treatment on days 1 −7 promoted cardiomyocyte differentiation. Different doses of heparin (from 0.3 to 10 ug/ml) were tested for their effects on cardiomyocyte differentiation. As shown in FIG. 19 with cTnT staining, 1 ug/ml was identified as a particularly advantageous dosage of heparin for enhancement of cardiomyocyte differentiation.

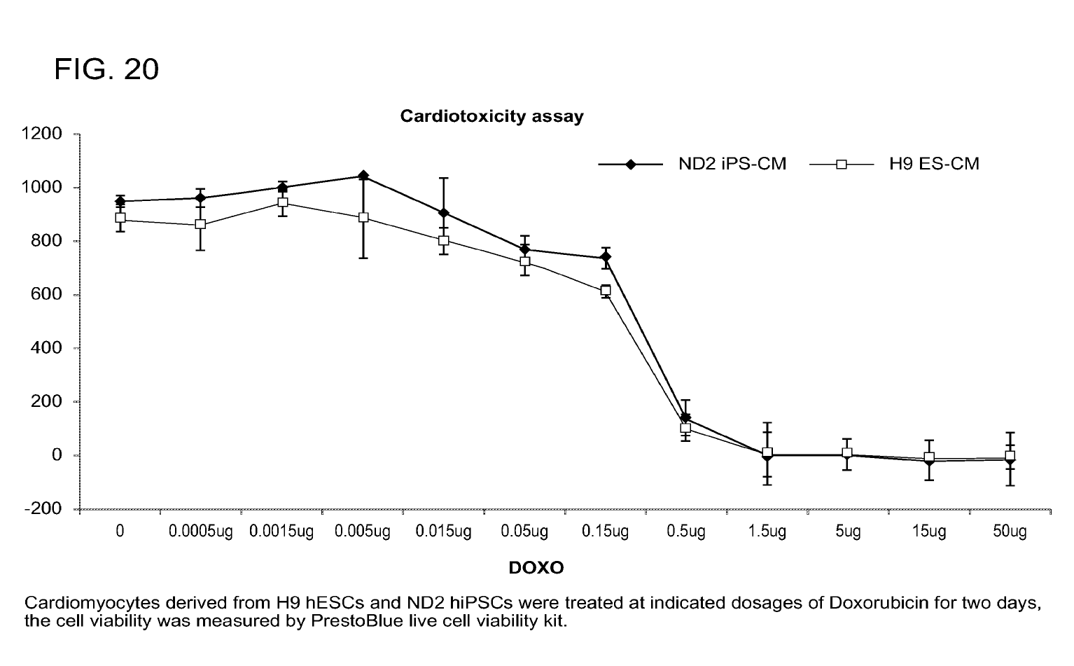

One application of this new approach using heparin and heparan sulfate to improve the production of cardiac differentiation from human ESC and iPSCs is in a cardiac toxicity assay, where a drug is administered to cardiac cells. FIG. 20 shows a doxycycline (Dox) killing curve. Cardiomyocytes were treated with doxocycline at indicated concentrations for 2 days.

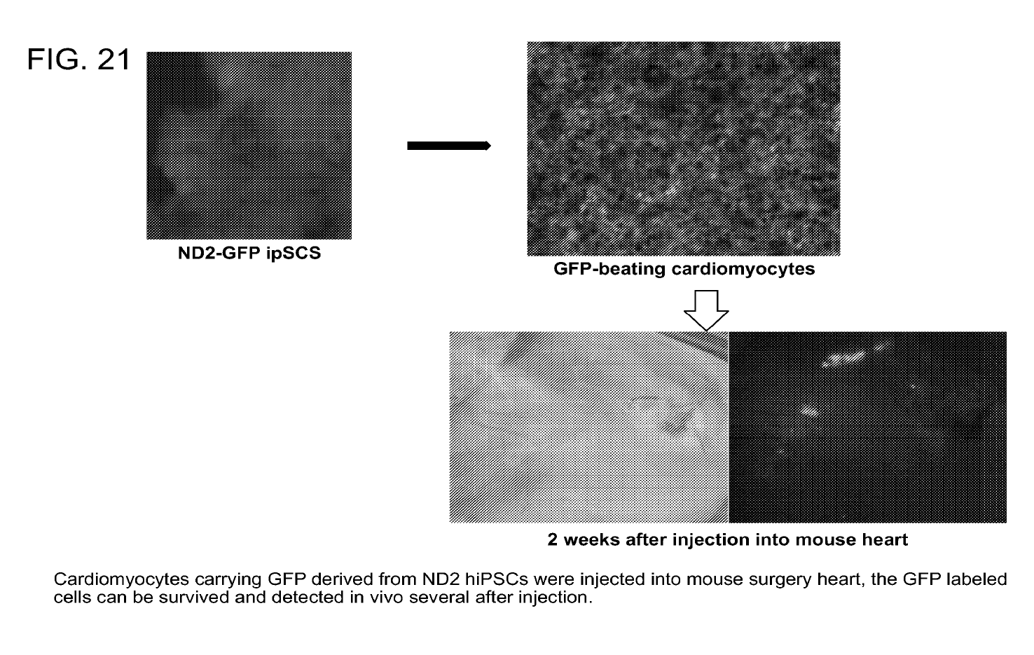

Another application is in transplantation. As shown in FIG. 21, human iPSC induced GFP-beating cardiomyocytes were generated. Two weeks after injection into the mouse heart, the GFP cells could be observed as beating. Even 5 weeks after injection, GFP was still visible in the heart (data not shown).

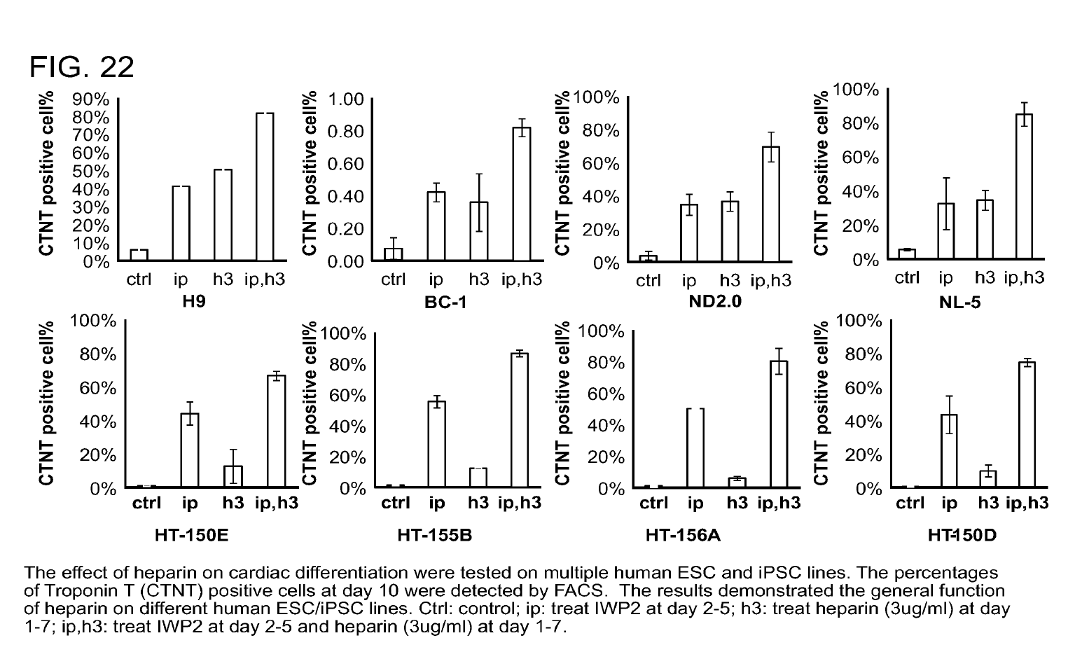

The general function of heparin on the following human ESC/iPSC lines was also demonstrated: H9, BC-1, ND2.0, NL-5, HT-150E, HT-155B, HT-156A and HT-150D (FIG. 22). In such experiments, the effect of heparin on cardiac differentiation was tested, using Troponin T (CTNT) as a marker. A positive effect of heparin was observed.

As described herein, the present invention allows for efficient production of cardiac cells for transplantation and drug discovery. Advantageously, the invention provides a fully defined system for cardiac differentiation, and it is suitable for clinical applications. The high efficiency and consistency in this invention are attractive and necessary for industrial-format mass production. This new discovery simplifies the culture system for cardiac differentiation, and could serve as a new platform for further technology development. The invention can also be used to produce clinical grade cardiac cells.

### INCORPORATION BY REFERENCE

Throughout this application, various patents, patent applications and publications are referenced. The disclosures of these publications in their entireties are hereby incorporated by reference to the same extent as if each independent patent and publication was specifically and individually indicated to be incorporated by reference.

What is claimed is:

1. A low protein medium that supports the proliferation and differentiation of stem cells comprising one or more of a volume expander, a lipid mix and a growth factor modulator.

2. The low protein medium of claim 1, wherein the volume expander comprises one or more polysaccharides, taurine or albumin.

3. The low protein medium of claim 1 or 2, wherein the one or more polysaccharides is selected from the group consisting of: trehalose, CM-Dextran or glycogen, beta-cyclodextrin, beta-hydrodextrin, N-Acetyl-Glucosamine, Methyl alpha-D-Glycogyrate, Methyl beta-D-glycogyrate, Dextrin 100K, Methyl-1-beta-cyclodextrin.

4. The low protein medium of any one of the preceding claims, wherein the lipid mix comprises: arachadonic acid, cholesterol, DL-alpha-tocopherol acetate, ethyl alcohol 100%, linoleic Acid, linolenic Acid, myristic acid, oleic acid, palmitic acid, palmitoleic acid, Pluronic F-68, stearic acid and tween-80.

5. The low protein medium of any one of the preceding claims, wherein the growth factor modulator is selected from the group consisting of: heparin and heparan sulfate.

6. The low protein medium of any one of the preceding claims, wherein the medium is albumin-free.

7. The low protein medium of any one of the preceding claims, wherein the medium comprises: water, salts, amino acids, vitamins, a carbon source, insulin, an FGF, selenium, transferrin, and one of TGF-β and NODAL, each in an amount sufficient to support pluripotent stem cell proliferation.

8. The low protein medium of any one of the preceding claims, wherein the stem cell is a totipotent, pluripotent, multipotent, oligopotent or unipotent stem cell.

9. The low protein medium of any one of the preceding claims, wherein the stem cell is an embryonic stem cell (ESCs), an induced pluripotent stem cell (iPSCs), a fetal stem cell or an adult stem cell.

10. The low protein medium of any one of the preceding claims, wherein the stem cell is a mammalian stem cell.

11. The low protein medium of claim 10, wherein the mammalian stem cell is a human stem cell.

12. The low protein medium of any one of the preceding claims, wherein the stem cell is a human embryonic stem cell or a human induced pluripotent stem cell.

13. The low protein medium of any one of the preceding claims, wherein the medium can support proliferation at high density.

14. The low protein medium of any one of the preceding claims, wherein the high density is confluency of 70% or more.

15. The low protein medium of any one of the preceding claims, comprising a volume expander.

16. The low protein medium of any one of the preceding claims, comprising a lipid mix and a growth factor modulator.

17. The low protein medium of any one of the preceding claims, wherein one or more growth factors are removed from the low protein medium.

18. The low protein medium of claim 17, wherein the growth factors are selected from the group consisting of: TGFβ, FGF and insulin.

19. The low protein medium of any one of claims 1-16, wherein one or more promoters of WNT signaling are added.

20. The low protein medium of any one of claims 1-16, wherein one or more inhibitors of WNT signaling are added.

21. A method for culturing stem cells, the method comprising the steps of:
placing pluripotent stem cells on a matrix or in suspension; and
contacting the cells with the medium of claim 1.

22. A method for deriving an induced pluripotent stem cell, the method comprising the steps of:
placing the pluripotent stem cells on a matrix or in suspension; and
contacting the cells with the medium of claim 1.

23. The method of claim 21 or 22, wherein the stem cell is a totipotent, pluripotent, multipotent, oligopotent or unipotent stem cell.

24. The method of claim 21 or 22, wherein the stem cell is an embryonic stem cell (ESCs), an induced pluripotent stem cell (iPSCs), a fetal stem cell or an adult stem cell.

25. The method of any one of claims 21-24, wherein the stem cell is a mammalian stem cell.

26. The method of claim 25, wherein the mammalian stem cell is a human stem cell.

27. The method of any one of claims 21-26, wherein the stem cell is a human embryonic stem cell or a human induced pluripotent stem cell.

28. A method for deriving a cardiac cell under defined conditions, the method comprising the step of:
culturing a pluripotent stem cell in the low protein medium of claim 1.

29. The method of claim 28, wherein the low protein medium comprises a lipid mix and a growth factor modulator.

30. The method of claim 28 or 29, wherein one or more promoters of WNT signaling are added.

31. The method of any one of claims 28-30, wherein one or more inhibitors of WNT signaling are added.

32. The method of any one of claims 28-31, wherein one or more growth factors are removed from the low protein medium.

33. The method of claim 32, wherein the growth factors are selected from the group consisting of: TGFβ, FGF and insulin.

34. A method for inducing differentiation of a cardiac cell in vitro, the method comprising:
obtaining a population of cells comprising a stem cell; and
contacting the population of cells with an amount of a low protein medium comprising one or more of a volume expander, a lipid mix and a growth factor modulator sufficient to induce differentiation of a cardiac cell from a stem cell of the population,
thereby inducing differentiation of a cardiac cell.

35. The method of claim 34, wherein the growth factor modulator is selected from the group consisting of heparin and heparan sulfate.

36. The method of claim 34 or 35, wherein the medium comprises at least about 0.3 μg/ml heparin or heparan sulfate.

37. The method of claim 34 or 35, wherein the medium comprises at least about 1 μg/ml heparin or heparan sulfate.

38. The method of claim 34 or 35, wherein the medium comprises heparin or heparan sulfate at about 1 μg/ml.

39. The method of any one of claims 34-38, wherein the medium is supplemented with insulin.

40. The method of any one of claims 34-39, wherein the contacting occurs on days 1-7 of culture.

41. The method of any one of claims 34-40, wherein the medium is albumin-free.

42. The method of any one of claims 34-41, wherein the medium comprises water, salts, amino acids, vitamins, a carbon source, insulin, an FGF, selenium, transferrin, and one of TGF-β and NODAL, each in an amount sufficient to support pluripotent stem cell proliferation.

43. The method of any one of claims 34-42, wherein the stem cell is an embryonic stem cell (ESCs), an induced pluripotent stem cell (iPSCs), a fetal stem cell or an adult stem cell.

44. The method of any one of claims 34-43, wherein the stem cell is a mammalian stem cell.

45. The method of claim 44, wherein the mammalian stem cell is a human stem cell.

46. The method of any one of claims 34-45, wherein the population of cells is at high density.

47. The method of claim 46, wherein the high density is confluency of 70% or more.

48. The method of any one of claims 34-47, wherein the medium comprises one or more polysaccharides selected from the group consisting of: trehalose, CM-Dextran or glycogen, beta-cyclodextrin, beta-hydrodextrin, N-Acetyl-Glucosamine, Methyl alpha-D-Glycogyrate, Methyl beta-D-glycogyrate, Dextrin 100K and Methyl-1-beta-cyclodextrin.

49. The method of any one of claims 34-48, wherein the medium comprises a growth factor selected from the group consisting of TGFβ, FGF and insulin.

50. A cardiac cell prepared by the method of claim 34.

51. A method for performing a cardiac toxicity assay comprising contacting a cardiac cell of claim 50 with a drug, and monitoring the function or survival of the cardiac cell.

52. A method for transplanting a cardiac cell into a subject comprising:
obtaining a cardiac cell prepared by the method of claim 34; and
administering the cardiac cell to the subject,
thereby transplanting a cardiac cell into the subject.

53. The method of claim 52, wherein the cardiac cell is administered to the heart of the subject.

54. The method of claim 52 or 53, wherein the subject is human.

55. The method of any one of claims 52-54, wherein the subject has heart disease.

56. The method of any one of claims 52-55, wherein the population of cells obtained to produce the cardiac cell is a population of cells of the subject.

57. A kit comprising a low protein medium that supports the proliferation and differentiation of stem cells comprising one or more of a volume expander, a lipid mix and a growth factor modulator.

58. The kit of claim 57, further comprising directions for the use of the medium for culturing stem cells or deriving an induced pluripotent stem cell.
